(19) United States
(12) Patent Application Publication
(10) Pub. No.: US 2019/0292559 A1
(43) Pub. Date: Sep. 26, 2019

(54) NUCLEOTIDE SEQUENCE FOR IMPROVING RESISTANCE AGAINST PLANT PATHOGENS

(71) Applicant: CONSEJO SUPERIOR DE INVESTIGACIONES CIENTIFICAS (CSIS), Madrid (ES)

(72) Inventors: Selena GIMENEZ IBANEZ, Madrid (ES); Roberto SOLANO TAVIRA, Madrid (ES); Marta BOTER GIL, Madrid (ES); Andrea CHINI, Madrid (ES); Gloria GARCIA CASADO, Madrid (ES); Andres ORTIGOSA URBIETA, Madrid (ES)

(21) Appl. No.: 16/347,979

(22) PCT Filed: Nov. 7, 2017
(86) PCT No.: PCT/EP2017/078493

§ 371 Date: May 7, 2019

(30) Foreign Application Priority Data
Nov. 7, 2016 (EP) 16382513.6

(51) Int. Cl. C12N 15/82; C07K 14/415

(52) U.S. Cl. CPC C12N 15/8281; C07K 14/415

(57) ABSTRACT

The invention relates to a nucleic acid sequence that improves resistance to biotrophic pathogens, in particular to Pseudomonas syringae, without affecting susceptibility to necrotrophs, in particular Botrytis cinerea. The present invention also relates to a plant that comprises the nucleic acid of the invention and a method to generate plants resistant to biotrophic pathogens and necrotrophs.

The invention relates to a nucleotide sequence that improves resistance to biotrophic pathogens, in particular to Pseudomonas, preferably to P. syringae, without increasing susceptibility to necrotrophs. Therefore, the present invention can be circumscribed to the agricultural field.

### BACKGROUND ART

In plants, preformed physical and biochemical barriers constitute the first line of plant defence against pathogens. Phytopathogenic bacteria must enter by natural surface openings such as stomata or wounds. To defend themselves, plants have evolved sophisticated strategies to perceive their attacker during the infection process and to translate this perception into an effective immune response. Two tiers of recognition by the innate immune system have been defined (Jones, J. D. and Dangl, J. L., Nature 2006; 444(7117): 323-329). One of them is triggered by the recognition of highly conserved microbe-associated molecular patterns (MAMPs) by host cell transmembrane proteins that function as pattern recognition receptors (PRRs), which in turn, activate MAMP-triggered immunity (MTI; (Jones, J. D. and Dangl, J. L. Nature 2006; 444(7117): 323-329)). This activates sufficient defence to resist non-pathogenic microbes and probably also, some pathogens. Plants rapidly close stomata upon perception of MAMPs during pathogen infection to inhibit the entry of pathogen and host tissue colonization (Melotto, M., et al. Cell. 2006; 126(5): 969-980). Weak phytobacterial epiphytics that fail to enter leaf tissues die on the surface of the leave, where conditions are supposed to be difficult for bacterial survival. The bacterial-triggered stomatal closure response, so-called the stomatal defence layer, represents an integral part of plant innate immune system.

Pseudomonas syringae (P. syringae) is a widespread bacterial pathogen that causes disease on a broad range of economically important plant species. In order to infect, P. syringae produces a number of phytotoxins and introduces virulence effector proteins into the plant cell to promote plant susceptibility (Jones, J. D. and Dangl, J. L.. Nature 2006; 444: 323-329; O′Brien, H. E. et al. Annu Rev Phytopathol. 2011; 49: 269-289; Xin, X. F. and He S. Y. Annu Rev Phytopathol. 2013; 51: 473-498). Some P. syringae strains have evolved a sophisticated strategy for manipulating the complex hormonal homeostasis in which plant immunity relies on, by producing coronatine (COR), a mimic of the bioactive jasmonic acid (JA) hormone: JA-isoleucine (JA-Ile) (Fonseca, S. et al. Curr Opin Plant Biol. 2009; 12(5): 539-547). In general terms, salicylic acid (SA) signaling mediates resistance against biotrophic and hemi-biotrophic microbes such as P. syringae, whereas a combination of JA and ethylene (ET) pathways activates resistance against necrotrophs such as the fungal pathogen Botrytis cinerea (Robert-Seilaniantz, A. et al. Annu Rev Phytopathol. 2011; 49: 317-343). SA and JA/ET defence pathways generally antagonize each other and thus, elevated resistance against biotrophs is often correlated with increased susceptibility to necrotrophs, and vice versa (Grant, M. and Lamb, C. Curr Opin Plant Biol. 2006; 9(4):414-20). COR contributes to disease symptomatology by inducing chlorotic (yellow) lesions (Kloek, A. P. et al. Plant J. 2001; 26(5): 509-522, Brooks, D. M. et al. Mol Plant Microbe Interact. 2004; 17(2): 162-174; Uppalapati, S. R. et al. Mol Plant Microbe Interact. 2007; 20(8): 955-965), facilitates entry of the bacteria into the plant host by stimulating the re-opening of stomata after bacterial-triggered stomatal closure (Melotto, M. et al. Cell. 2006; 126(5): 969-980, Melotto, M. et al. Annu Rev Phytopathol. 2008; 46: 101-122), promotes bacterial growth in the apoplast by inhibiting SA-dependent defences required for P. syringae resistance, because of its activation of the antagonistic JA pathway (Cui, J. et al. Proc Natl Acad Sci USA. 2005; 102(5): 1791-1796; Laurie-Berry, N. et al. Mol Plant Microbe Interact. 2006; 19(7): 789-800) and enhances bacterial virulence systemically.

JAZ co-receptors are COI1 (COI1 coronatine-insensitive protein 1) substrates that negatively regulate the JA-signalling pathway by directly interacting with and repressing MYC2-like transcription factors (TFs) that control JA-regulated genes (Chini, A. et al. Nature. 2007; 448(7154): 666-671; Thines, B. et al. Nature. 2007; 448(7154): 661-665; Sheard, L. B. et al. Nature. 2010; 468(7322): 400-405; Fernandez-Calvo, P. et al. Plant Cell. 2011; 23(2): 701-715; Pauwels, L. and Goossens, A. Plant Cell. 2011; 23(9): 3089-3100). Repression of TFs by JAZs is mediated by a general co-repressor machinery (Pauwels, L. et al. Nature. 2010; 464(7289): 788-791). The JAZ family of JA-repressors have emerged as central modulators of JA signaling (Chini, A. et al. Nature. 2007; 448(7154): 666-671; Thines, B. et al. Nature. 2007; 448(7154): 661-665; Yan, Y. et al. Plant Cell. 2007; 19(8): 2470-2483). The JAZ family consists of 13 members in Arabidopsis showing different tissue- and stage-specific expression patterns (Chini, A. et al. Nature. 2007; 448(7154): 666-671; Thines, B. et al. Nature. 2007; 448(7154): 661-665; Yan, Y. et al. Plant Cell. 2007; 19(8): 2470-2483).

In spite of the efforts made in order to minimize pathogen infection in plants by manipulating the master defence JA and SA hormonal pathways, current approaches have been unsuccessful due to the well-known antagonism between these pathways. This has been mostly due to the fact that antagonistic interactions between SA and JA pathways would be detrimental to some pathogens but beneficial to others, all of which are present in the field. Consequently, elevated resistance against biotrophs is often correlated with increased susceptibility to necrotrophs, and vice versa (Grant, M. and Lamb, C. Curr Opin Plant Biol. 2006; 9(4):414-20). For instance, dominant mutant versions of JAZ repressors without the Jas domain (JAZdeltaJas) promote JA-insensitivity and, as a consequence, increase resistance to biotrophs but increase susceptibility to necrotrophs (Chini, A. et al. Nature. 2007; 448(7154): 666-671; Thines, B. et al. Nature. 2007; 448(7154): 661-665; Chung, H.S. et al. Phytochemistry. 2009,70(13-14):1547-59). Thus, there is an urgent need to obtain an effective method to protect plants against both biotrophs and necrotrophs, or at least to protect plants against one type of pathogens without increasing the susceptibility to the other type, by overcoming the antagonistic relationship between JA-SA master hormonal defense pathways, that could be realistically transferred to protect crop plants in the field.

### SUMMARY OF THE INVENTION

In the present invention is demonstrated that JAZ2, a family member of the jasmonate zim-domain (JAZ) proteins, is constitutively expressed in stomata guard cells and is required for stomatal closure after bacterial recognition. Additionally, it is also show that in addition to MYC2, MYC3 and MYC4 are also repressed by JAZ2 and regulate the expression of ANAC19, ANAC55 and ANAC72 to modulate stomata aperture. Therefore, the results of the present invention demonstrate the existence of a COI1-JAZ2-MYC2,3,4-ANAC19,55,72 module responsible for the regulation of stomatal aperture that is hijacked by bacterial COR to promote infection. More importantly, dominant mutations that make JAZ2 resistant to degradation (hereafter called JAZ2deltaJas or JAZ2ΔJas forms) fully prevent stomatal re-opening by COR and are highly resistant to biotrophs and/or hemi-biotrophs, such as P. syringae, and additionally these plants do not show an increase of susceptibility to necrothophs, such as Botrytis cinerea, Plectosphaerella cucumerina and/or Alternaria brassicicola. Consequently, the inventors solve the trade-off of the antagonics interactions between the SA and JA defense pathway, in which an increase in the resistance to biotrophic pathogens (due to JA-insensitivity/by blocking the JA-signaling pathway) results in an enhanced susceptibility to necrotrophs, and vice versa.

The key issue of this invention is that the JA-dependent signalling is specifically modified at stomata without affecting JA responses at plant leaves (mesophyll cells) and, therefore, without affecting susceptibility to general necrotrophic pathogens, only restricting the entry of biotrophic pathogens that use the stomata as entry ports for invasion. The use in crops of the guard cell specific expression of a modified JAZ nucleic acid, operably linked to a promoter that regulates the specific expression of the nucleic acid to the guard cells of the stomata, wherein said modified JAZ encodes a polypeptide comprising a ZIM domain, but not a functional Jas motif, has the potential to solve the trade-off between resistance to biotrophs and necrotrophs as it uncouples spatially the SA-JA antagonism controlling resistance to both types of pathogens. Solving this trade-off has been a major issue in agriculture.

Remarkably, the above-described modified JAZ2 sequence (hereafter called JAZ2deltaJas) that encodes a JAZ2 variant resistant to degradation confers improved resistance to the biotrophic and/or hemi-biotrophic pathogens, such as Pseudomonas syringae, without altering levels of resistance against a broad range of necrotrophic pathogenic fungi such as Botrytis cinerea, P. cucumerina and/or A. brassicicola, which do not use stomata to enter the host. This discovery also evidences novel cell type specific strategies for crop protection against bacterial biotrophic infections that use stomata as entry ports, without compromising whole leaf resistance to necrotrophic pathogens. This invention is based on the fact that generally, pathogenic biotrophic bacteria enter through stomatal pores while fungal necrotrophic pathogens use direct penetration of the plant leave surface to enter the host by localized secretion of lytic enzymes and/or mechanical force.

In the examples included in the present invention is demonstrated that JAZ2 is a specific JAZ located at the stomata, and that inactivation or deletion of “Jas” domain (domain Jasmonate-specific) of JAZ2 in plants improve resistance to biotrophic and/or hemi-biotrophic pathogens, such as Pseudomonas syringae bacteria while also maintaining the resistance to necrotrophs such as Botrytis cinerea or any other necrothropic pathogens disclosed in the present invention. As pointed above, dominant mutant versions of JAZ repressors without the functional Jas domain (JAZdeltaJas) promote JA-insensitivity and, as a consequence, increase resistance to biotrophs but also increase susceptibility to necrotrophs (Chini, A. S. et al. Nature. 2007; 448(7154): 666-671; Thines, B. et al. Nature. 2007; 448(7154): 661-665; Chung, H. S. et al. Phytochemistry. 2009,70(13-14):1547-59). The key invention of this work is that combining the inactivation (non-functional) or deletion of the Jas domain described in the present invention together with the specific expression of the modified JAZ sequence at the guard cells of the stomata, results in an increased resistance of the plant to biotrophs, but without the trade-off of enhancing susceptibility to necrotrophs, because the JA-insensitivity promoted by JAZdeltaJas is restricted to guard cells and do not affect mesophyll cells defence.

The JAZ2ΔJas mutant protein does not interact with COI1, and therefore this mutant protein is resistant to proteasome-mediated degradation, so that the resulting plants are insensitive to JA in the stomata. Inventors have conducted comparative experiments with another mutant of the same family, JAZ1 and JAZ3 (eliminating the Jas domain as well) finding that the JAZ2 truncated form or JAZ2 comprising a non-functional or inactivated “Jas domain” is surprisingly the one that provides better results for resistance to both types of pathogens due to the specific presence at the guard cells of the stomata of the JAZ2 truncated protein or JAZ2 with a non-functional “Jas domain” but not of the JAZ1 or JAZ3 truncated forms, which are expressed in other tissues.

The JAZ family of JA-repressors are characterized by two highly conserved sequence motifs, namely ZIM (central) and Jas (C-terminal) domains, containing almost all JAZs the functional domains required for function. This indicates that the specific activity of modified JAZ2 protein in restricting bacterial biotrophic invasion compared to any other JAZ is due the specific targeting of JAZ2 to the stomata. Thus, besides the use of JAZ2ΔJas variants that are naturally present at the stomata, it was also establish the use of any other JAZΔJas variant (from all existing JAZ proteins, i.e. JAZ1 to JAZ13) artificially targeted to the stomata through the use of new promoters or modification of natural promoters that would led to expression at guard cells. Experiments with these truncated JAZ proteins have been made in Arabidopsis thaliana and it is expected that the same technical effect will be achieved in all plant that have stomata, e.g. tomato, potato, tobacco, canola, rice and in general all higher plants.

Interestingly for the products obtained by the enterprises of the breeding sector, plant mutants can be generated in cultures without transgenesis (e.g. by gene editing techniques such as CRISPR/Cas).

A first aspect of the invention refers to an isolated nucleic acid sequence that comprises a modified JAZ nucleic acid, operably linked to a promoter that regulates the specific expression of the nucleic acid in the guard cells of the stomata, wherein said modified JAZ encodes a polypeptide comprising a ZIM domain, but not a functional Jas domain; a functional variant of the modified JAZ nucleic acid or JAZ polypeptide (JAZ/JAZ), a homologue or orthologue thereof. Preferably the modified JAZ, or the corresponding modified JAZ polypeptide, is selected from the list consisting of any modified JAZ1, JAZ2, JAZ3, JAZ4, JAZ5, JAZ6, JAZ7, JAZ8, JAZ9, JAZ10, JAZ11, JAZ12 and JAZ13 nucleic acids; a functional variant of any modified JAZ1, JAZ2, JAZ3, JAZ4, JAZ5, JAZ6, JAZ7, JAZ8, JAZ9, JAZ10, JAZ11, JAZ12 and JAZ13 nucleic acid; a homologue of any modified JAZ1, JAZ2, JAZ3, JAZ4, JAZ5, JAZ6, JAZ7, JAZ8, JAZ9, JAZ10, JAZ11, JAZ12 and JAZ13; or orthologue of any modified JAZ1, JAZ2, JAZ3, JAZ4, JAZ5, JAZ6, JAZ7, JAZ8, JAZ9, JAZ10, JAZ11, JAZ12 and JAZ13.

The acronym JAZ refers to “Jasmonate ZIM-Domain”. All JAZ proteins contain two highly conserved sequence motifs, the ZIM domain in the central portion of the protein, and the C-terminal Jas motif. In addition, there is a weakly conserved region at the N-terminus. Wide explanations can be found at scientific bibliography to know the function of said domains and motifs.

From now on the isolated nucleic acid of the first aspect of the present invention will be also referred to as the “nucleic acid of the invention”. Herein the terms “nucleic acid of the invention”, “nucleotide sequence of the invention”, “polynucleotide of the invention” and “oligonucleotide of the invention” are used interchangeably.

The modified (non-naturally occurring) JAZ nucleic acid of the first aspect of the invention comprises any known JAZ nucleic acids, with the proviso that comprise the ZIM domain but not a functional Jas domain.

In a particular embodiment of the present invention the modified JAZ nucleic acid or the corresponding modified JAZ polypeptide is from Arabidopsis thaliana (A. thaliana; At). Therefore, the modified JAZ, or the corresponding modified JAZ polypeptide, is selected from the list consisting of any modified AtJAZ1, AtJAZ2, AtJAZ3, AtJAZ4, AtJAZ5, AtJAZ6, AtJAZ7, AtJAZ8, AtJAZ9, AtJAZ10, AtJAZ11, AtJAZ12 and AtJAZ13 nucleic acids from A. thaliana that comprise the ZIM domain but not a functional Jas domain; a functional variant of any modified AtJAZ1, AtJAZ2, AtJAZ3, AtJAZ4, AtJAZ5, AtJAZ6, AtJAZ7, AtJAZ8, AtJAZ9, AtJAZ10, AtJAZ11, AtJAZ12 and AtJAZ13 nucleic acid; a homologue of any modified AtJAZ1, AtJAZ2, AtJAZ3, AtJAZ4, AtJAZ5, AtJAZ6, AtJAZ7, AtJAZ8, AtJAZ9, AtJAZ10, AtJAZ11, AtJAZ12 and AtJAZ13; or orthologue of any modified AtJAZ1, AtJAZ2, AtJAZ3, AtJAZ4, AtJAZ5, AtJAZ6, AtJAZ7, AtJAZ8, AtJAZ9, AtJAZ10, AtJAZ11, AtJAZ12 and AtJAZ13.

In A. thaliana the JAZ genes codify for the following JAZ proteins:

JAZ1 gene codifies for JAZ1 protein: Locus At1g19180 (JAZ1 iHOPTIFY10A iHOP, ATJAZ1, JASMONATE-ZIM-DOMAIN PROTEIN 1, TIFY10A).

JAZ2 gene codifies for JAZ2 protein: Locus At1g74950 (JASMONATE-ZIM-DOMAIN PROTEIN 2, TIFY10B). SEQ ID NO: 1 refers to its cDNA, SEQ ID NO: 2 to the genomic DNA and SEQ ID NO: 3 to the protein.

JAZ3 gene codifies for JAZ3 protein: Locus At3g17860 (JA13, JASMONATE-INSENSITIVE 3, JASMONATE-ZIM-DOMAIN PROTEIN 3, TIFY6B).

JAZ4 gene codifies for JAZ4 protein: Locus At1g48500 (ATJAZ4, JASMONATE-ZIM-DOMAIN PROTEIN 4, TI FY DOMAIN PROTEIN 6A, TIFY6A).

JAZ5 gene codifies for JAZ5 protein: Locus At1g17380 (JASMONATE-ZIM-DOMAIN PROTEIN 5, TIFY11A).

JAZ6 gene codifies for JAZ6 protein: At1g72450 (JASMONATE-ZIM-DOMAIN PROTEIN 6, TI FY DOMAIN PROTEIN 11B, TI FY11B).

JAZ7 gene codifies for JAZ7 protein: Locus At2g34600; Gene ID: 818025; (JASMONATE-ZIM-DOMAIN PROTEIN 7, TIFY5B).

JAZ8 gene codifies for JAZ8 protein: Locus At1g30135; GenBank: ABG48454.1 (JASMONATE-ZIM-DOMAIN PROTEIN 8, TIFY5A).

JAZ9 gene codifies for JAZ9 protein: Locus At1g70700 (JASMONATE-ZIM-DOMAIN PROTEIN 9, TIFY7).

JAZ10 gene codifies for JAZ10 protein: Locus At5g13220 (JAS1, JASMONATE-ASSOCIATED 1, JASMONATE-ZIM-DOMAIN PROTEIN 10, TIFY DOMAIN PROTEIN 9, TIFY9).

JAZ11 gene codifies for JAZ11 protein: Locus At3g43440 (JASMONATE-ZIM-DOMAIN PROTEIN 11, TIFY3A).

JAZ12 gene codifies for JAZ12 protein: Locus At5g20900 (JASMONATE-ZIM-DOMAIN PROTEIN 12, TIFY3B).

JAZ13 gene codifies for JAZ13 protein: Locus At3g22275 (JASMONATE ZIM-DOMAIN PROTEIN 13).

A preferred embodiment of the first aspect of the invention relates to the isolated nucleic acid wherein the ZIM domain comprises the sequence SEQ ID NO: 4 preferably the ZIM domain comprises a sequence with at least 65% identity with SEQ ID NO: 5; a functional variant of the modified JAZ/JAZ, a homologue or orthologue thereof.

The ZIM domain is conserved among different plant species and it is a sequence well known by the expert in the field. The SEQ ID NO: 4 relates to a consensus sequence when compared the sequences shown in the table:

ZIM domain

| SEQ ID | Protein | Selected ZIM domain sequence |
|---|---|---|
| SEQ ID NO: 16 | AtJAZ1 | PLTIFYAGQVIVFNDFSAEKAKEVINLA |
| SEQ ID NO: 5 | AtJAZ2 | PLTIFYGGRVMVFDDFSAEKAKEVIDLA |
| SEQ ID NO: 17 | AtJAZ3 | QLTIFYAGSVCVYDDISPEKAKAIMLLA |
| SEQ ID NO: 18 | AtJAZ4 | QLTIFYAGSVLVYQDIAPEKAQAIMLLA |
| SEQ ID NO: 19 | AtJAZ5 | QLTIFFGGKVLVYNEFPVDKAKEIMEVA |
| SEQ ID NO: 20 | AtJAZ6 | QLTIFFGGKVMVFNEFPEDKAKEIMEVA |
| SEQ ID NO: 21 | AtJAZ7 | ILTIFYNGHMCVSSDLTHLEANAILSLA |
| SEQ ID NO: 22 | AtJAZ8 | RITIFYNGKMCFSSDVTHLQARSITSIA |
| SEQ ID NO: 23 | AtJAZ9 | QLTIFYGGTISVFNDISPDKAQAIMLCA |
| SEQ ID NO: 24 | AtJAZ10 | PMTIFYNGSVSVFQVSRNKAGEIMKVA |
| SEQ ID NO: 25 | AtJAZ11 | QLTIIFGGSFSVFDGIPAEKVQEILHIA |
| SEQ ID NO: 26 | AtJAZ12 | QLTIFFGGSVTVFDGLPSEKVQEILRIA |

The percentage of identity between the ZIM domain of the JAZ2 protein of A. thaliana (SEQ ID NO: 5) and other ZIM-domain containing proteins (TIFY-like proteins) from other plants is the following: 71% for Setaria italica, 68% for Malus domestica and 78% for Nelumbo nucifera.

Preferably, the ZIM domain of the nucleic acid of the invention comprises SEQ ID NO: 27 (TIFY sequence wherein F and/or Y amino acids are substituted by conserved amino acids). More preferably, the ZIM domain comprises SEQ ID NO: 28 (TIFY sequence).

The term “identity”, as used herein, refers to the percentage of identical nucleic acid residues between two nucleic acid sequences when they are compared. The sequence comparison methods are known in the art, and include but are not limited to BLASTN, and FASTA program ClustalW. The expert in the field can consider that nucleic acid identity percentages of at least 70%, 80%, 85%, 90%, 95%, 96%, 97%, 98% or 99% maintain the same properties and functions of said nucleic acid.

In the present invention when the ZIM domain comprises a sequence with at least 65% identity with SEQ ID NO: 5; it means that the percentage of identity can be 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%.

A more preferred embodiment of the first aspect of the invention relates to the isolated nucleic acid wherein the Jas motif (domain) comprises the sequence SEQ ID NO: 6, preferably comprises a sequence with at least 70% identity with SEQ ID NO: 7; a functional variant of the modified JAZ nucleic acid, a homologue or orthologue thereof.

As used herein the term “non-functional”, “inactivated” or “inactivation” when referring to a Jas domain (motif) means that the known normal function or activity of this domain has been eliminated or highly diminished. In this sense, wherein these terms are binding to Jas domain means that JAZdeltaJas proteins confer JA-insensitivity in the plants. The residues of the Jas domain involved in the interaction with COI1 and JA-Ile have been described in Sheard et al (Sheard, L. B. et al. Nature. 2010 (468): 400-405). As used herein, any mutation in one or more of these essential residues should eliminate JAZ binding to COI1 and, thus, render the JAZ mutant resistant to degradation giving rise to a similar phenotype than JAZdeltaJas proteins. Additionally, inactivation which renders the gene or protein, preferably Jas domain, non-functional includes such methods as deletions, mutations, substitutions, interruptions or insertions in the nucleic acid gene sequence.

A “deletion” of a gene as used herein may include deletion of the entire coding sequence, deletion of part of the coding sequence, deletion of the regulatory region, deletion of the translational signals or deletion of the coding sequence including flanking regions.

As used herein the term “mutation” when referring to a nucleic acid refers to any alteration in a nucleic acid such that the product of that nucleic acid is partially or totally inactivated. Examples of mutations include but are not limited to point mutations, frame shift mutations and deletions of part or all of a gene.

As used herein, the term “modified” when referring to nucleic acid or a polynucleotide means that the nucleic acid has been altered in some way as compared to a wild-type, or a naturally-occurring nucleic acid, such as by mutation in; deletion of part or all of the nucleic acid; or by being operably linked to a transcriptional control region.

The Jas domain is conserved among different plant species and it is a sequence well known by the expert in the field (for example in Thireault, C. et al. The Plant Journal. 2015; 82:669-679). The SEQ ID NO: 6 relates to a consensus sequence when AtJAZ1-12 were compared according to Thireault, C. et al. (Theriault C. et al. The Plant Journal. 2015; 82:669-679).

As it is mentioned previously, JAZdeltaJas proteins disclosed in the present invention, which confer JA-insensitivity have a Jas domain (motif) eliminated, mutated or non-functional. In this sense, Sheard et al. (Sheard, L. B. et al. Nature. 2010 (468): 400-405) disclosed that the Jas domain sequence is sufficient for COI1 binding in the presence of the hormone. Consequently, any mutation performed in the at least one essential residues of the Jas domain comprising the SEQ ID NO: 6 or 7 of the present invention, which blocks the interaction with JA-Ile or COI1 are comprised in the present invention. Amino acids essential for the interaction of the Jas motif with COI1 or JA-Ile are described by Sheard L.B. (Sheard, L. B. et al. Nature. 2010; 468(7322): 400-405) for JAZ1.

The percentage of identity between the Jas domain of the AtJAZ2 protein (SEQ ID NO: 7) and other Jas-domain containing proteins from other plants is the following: 86% for Hevea brasiliensis and 88% for Vitis vinifera.

In the present invention when the Jas domain comprises a sequence with at least 80% identity with SEQ ID NO: 7; it means that the percentage of identity can be 80, 81, 82, 83, 84, 85, 86, 87, 88, 89, 90, 91, 92, 93, 94, 95, 96, 97, 98, 99 or 100%.

The nucleic acid sequence described in the first aspect of the present invention can be of any length, preferably between 2 and 1101 nucleotides, more preferably between 2 and 888 nucleotides. In a preferred embodiment the length of nucleic acid is 84 nucleotides.

When the modified JAZ nucleic acid is JAZ2 of A. thaliana, a preferred embodiment refers to the isolated nucleic acid that comprises the SEQ ID NO: 1 of any length that comprises at least nucleotides 1-438, preferably 1-603, and has deleted at least nucleotides 604-684. Another preferred embodiment refers to the isolated nucleic acid that comprises the SEQ ID NO: 2 of any length that comprises at least nucleotides 1-849, preferably 1-1101, and has deleted at least nucleotides 1102-1274. In all cases, there will be a minimal N-terminal size that contains the TIFY domain (Or variant thereof as described in SEQ ID NO: 16 to SEQ ID NO: 28) for recruiting NINJA/TOPLESS repressive machinery.

A more preferred embodiment of the first aspect of the present invention relates to the isolated nucleic acid which comprises any of the JAZ nucleic acids disclosed in the present invention selected from the native JAZ1 and JAZ3 to JAZ13, comprising a non-functional Jas domain, without their native promoter and with the condition that are operably linked to a promoter that regulates the specific expression of the nucleic acid in the guard cells of the stomata. In a preferred embodiment of the first aspect of the present invention, the promoter that regulates the specific expression of the nucleic acid in the guard cells of the stomata are selected from the group consisting of promoter of JAZ2 from A. thaliana (SEQ ID NO: 11), any regulatory sequence driving gene expression to the stomata such as MYB60 and GC1 among others (Yang, Y., et al. Plant Methods. 2008; 4: 6; Rusconi F., et al. J Exp Bot. 2013; 64(11):3361-71), preferably the promoter is the native promoter of JAZ2 from A. thaliana (SEQ ID NO: 11).

A more preferred embodiment of the first aspect of the invention relates to the isolated nucleic acid which comprises a sequence with at least 70% identity with SEQ ID NO: 8, corresponding to the cDNA of the modified AtJAZ2 (JAZ2ΔJas), or SEQ ID NO: 9, corresponding to the gDNA of the modified AtJAZ2 (JAZ2ΔJas); a functional variant, a homologue or orthologue thereof. In the present invention the term JAZ2ΔJas as synonymous of SEQ ID NO: 8 or 9 is used. The term JAZ2ΔJas is also used as a synonymous of SEQ ID NO: 10.

Herein the terms “nucleotide sequence”, “polynucleotide”, “nucleic acid” and “oligonucleotide” are used interchangeably.

The nucleotide sequence of the present invention is operably linked to a promoter or regulatory sequence that regulates the specific expression of the nucleic acid in the guard cells of the stomata. Said promoter is selected from any plant promoter regulating the expression of the nucleic acid of the invention in the guard cells of the stomata, or from any artificial promoter regulating the expression of said nucleic acid in the guard cells of the estomata. A more preferred embodiment of the first aspect of the invention relates to the isolated nucleic acid of the first aspect of the invention wherein the promoter is selected from the list consisting of: promoter of JAZ1, JAZ2, JAZ3, JAZ4, JAZ5, JAZ6, JAZ7, JAZ8, JAZ9, JAZ10, JAZ11, JAZ12 and JAZ13, preferably from A. thaliana. More preferably the promoter comprising a nucleic acid sequence, wherein said nucleic acid sequence has at least 80% sequence identity to the promoter of JAZ2 from A. thaliana (SEQ ID NO: 11); it means that the percentage of identity can be 80, 81, 82, 83, 84, 85, 86, 87, 88, 89, 90, 91, 92, 93, 94, 95, 96, 97, 98, 99 or 100%. A more preferred embodiment the promoter is the promoter of JAZ2 from A. thaliana (SEQ ID NO: 11). It could be also used any regulatory sequence driving gene expression to the stomata such as MYB60 and GC1 promoters among others (Yang, Y. et al. Plant Methods. 2008; 4: 6; Rusconi F. et al. J Exp Bot. 2013; 64(11):3361-71).

As it is used here, the term “promoter” refers to a region of the DNA upstream from the start point of the transcription of a gene, and particularly, that is able to start the transcription of a gene in a plant cell. Promoters contain specific DNA sequences such as response elements that provide an initial binding site for RNA polymerase and for transcription factors that recruit RNA polymerase. It is recognized by the expert in the field that, a promoter or a regulatory sequence that regulates the specific expression of a nucleic acid has at least all elements essential for transcription of said nucleic acid, including, for example, a TATA box or transcription factor binding sites.

The promoter referred in the present invention is a native or non-native promoter that is functional in plant cells. Examples of promoters include but are not limited to, promoters obtained from plants, virus of plants, or bacteria that can express the nucleic acid of the present invention in plant cells as Agrobacterium or Rhizobium.

Promoters can be classified, for instance, as inducible and constitutive promoters. The promoter as used in the present invention can be an inducible regulatory element that confers conditional expression in the presence of an inducing agent, increasing the expression compared to the expression of the nucleic acid in the absence of said inducing agent.

The term “operatively linked” refers to a juxtaposition in which the components as well described have a relationship that enables them to work in the intentional way. A polynucleotide operatively linked to any regulatory element that controls the expression of said polynucleotide is linked in such a way that the expression of the coding sequence of the polynucleotide is achieved under conditions compatibles with the regulatory element. The regulatory element contains at least all elements or sequences essential for transcription of said polynucleotide, known by the expert in the field. Said regulatory element may also include a terminator sequence.

The nucleotide sequence of the invention, in addition to the coding sequence, it may have other elements, such as but not limited, introns, noncoding sequences at the 5′ or 3′, binding sites for ribosomes, stabilizing sequences, promoters, etc. These polynucleotides may also include further coding sequences for additional nucleic acids, which may be useful, for example, but not limited, to enhance stability of the peptide generated from it or to allow a better purification thereof.

To refer to any of the polynucleotide described in the above aspect and its preferred embodiments of the present invention, the term “polynucleotide of the invention” or “polynucleotide of the present invention” can be used.

The nucleic acid sequence of the present invention may have conservative substitutions, known to the skilled expert. Conservative substitutions lead to a polynucleotide that encodes the same peptide.

The term polynucleotide includes genomic DNA or DNA encoding double or single stranded, ribonucleic acid (RNA), any synthetic and genetically manipulated polynucleotide, and both sense and antisense strands. This includes single-chain molecules and double-stranded, such as DNA-DNA hybrids, DNA-RNA and RNA-RNA.

The nucleotide sequence of the invention can be obtained artificially by conventional cloning methods selection and widely known in the prior art.

The nucleic acid can form part of an expression cassette, comprising the nucleic acid of the invention operably linked to control elements of transcription and/or translation.

A second aspect of the invention relates to a vector comprising the isolated nucleic acid of the first aspect of the invention.

The term “vector”, as used herein, refers to a nucleic acid molecule that is capable of transferring nucleic acid sequences contained therein to a cell. Examples of recombinant vectors are linear DNA, plasmid DNA, modified viruses, adenovirus/adeno-associated viruses, and retroviral viral vectors, etc.; all widely described in the literature and which can be used following standard molecular biology techniques or purchased from vendors. Vectors may be introduced by any method known to the skilled artisan, for example, but without limiting, by transfection, transformation or infection of the host cells, such as, but not limited to, plant cells.

A third aspect of the invention relates to a protein encoded by the isolated nucleic acid of the first aspect of the invention. From now on, “the protein of the invention”.

When the protein of the third aspect of the invention is codified by the modified of A. thaliana, a preferred embodiment refers to the protein that comprises the SEQ ID NO: 3 of any length that comprises at least aminoacids 1-146 (containing the TIFY, sequence SEQ ID NO: 28), preferably 1-201, more preferably 1-158; and has deleted or non-functional at least residues 202-228.

A preferred embodiment of the third aspect of the invention refers to the protein as defined in SEQ ID NO: 10 (JAZ2ΔJas).

The invention also provides for variants, analogs, homologues or ortologues of the protein of the invention encoded by the isolated nucleic acid of the first aspect of the invention.

As known by the expert in the field, the present invention encompass not only an specific modified JAZ nucleic acid or JAZ polypeptide as described herein, but also functional variants or homologues thereof that do not affect the biological activity and function of the resulting protein.

It is well known in the art that alterations in a nucleic acid sequence which result in the production of a different but conservative amino acid at a given site that do not affect the functional properties of the resulting polypeptide. For example, conservative amino acid changes may be made, which although they alter the primary sequence of the peptide, do not normally alter its function. Conservative amino acid substitutions of this type are known in the art, e.g, changes within the following groups: glycine and alanine; valine, isoleucine and leucine; aspartic acid and glutamic acid; asparagine and glutamine; serine and threonine; lysine and arginine; or phenylalanine and tyrosine.

Modifications (which do not normally affect the primary sequence) include in vivo or in vitro chemical derivatization of the peptide, e. g., acetylation, carbonation or glycosylation.

Generally, variants or homologues of a particular nucleotide sequence or polypeptide of the invention may be determined by sequence alignment programs known in the art.

“Variants” of the modified JAZ/JAZ can differ from naturally occurring nucleic acids/polypeptides by codons encoding conservative amino acid sequence differences or by modifications, which do not affect sequence, or by both.

“Homologues” of modified JAZ/JAZ are nucleic acids or polypeptides descending from a common ancestral nucleic acid sequence. Homologous sequence are either orthologue or paralogue sequence. The term homologue used in the present invention also designates a modified JAZ/JAZ orthologue from different plant species. Homologous sequences are orthologous if they originated by vertical descent from a single gene of the last common ancestor, that is, the copies of a single gene/protein in the two resulting species with a common ancestor sequence are orthologous sequences. Homologous sequences are paralogous if they were originated by a duplication event within the genome of particular specie. That is, the duplication of a gene in an organism occupying two different positions in the same genome.

For example, homologues of JAZ could be modified in order to obtain the modified nucleic acid or the modified polypeptide of the present invention from any of the following, not limitating, list of nucleic acids:

Theobroma cacao JAZ3 (TCM_012913, TCM_011017), Theobroma cacao JAZ6 (TCM_029176), Theobroma cacao JAZ12 (TCM_006179, TCM_037429), Medicago truncatula JAZ (MTR_5g013530, MTR_2g042900, MTR_6g069870, MTR_4g124960, MTR_8g021380, MTR_1g031930, MTR_8g107300, MTR_5g013515, MTR_2g019190, MTR_5g013520, MTR_4g124950), Theobroma cacao JAZ10 (TCM_008419), Theobroma cacao JAZ8 (TCM_029461), Theobroma cacao JAZ1 (TCM_037495), Solanum lycopersicum JAZ3 (LOC100191114), Solanum lycopersicum JAZ1 (LOC100134911).

Homologues of JAZ protein could be modified in order to obtain the modified polypeptide of the present invention from the nucleic acid encoding any of the following, not !imitating proteins:

Catharanthus roseus JAZ (ACM89458, ACM89457), Salvia miltiorrhiza JAZ3 (AHK23660), Salvia miltiorrhiza JAZ2 (AHK23659), Nicotiana attenuata JAZ (AFL46177, AFL46176, AFL46175, AFL46174, AFL46173, AFL46172, AFL46171, AFL46170, AFL46169, AFL46168, AFL46167, AFL46166, AFL46165, AFL46178), Phaseolus lunatus JAZ (AIT38287, AIT38286, AIT38285, AIT38283, AIT38282, AIT38281, AIT38275), Sonneratia apetala JAZ (AFU90901), Sonneratia lanceolata JAZ (AFU90900), Sonneratia ovata JAZ (AFU90899), Sonneratia caseolaris JAZ (AFU90898), Sonneratia alba JAZ (AFU90897), Phaseolus lunatus JAZ (AIT38274, AIT38273.1, AIT38272), Solanum lycopersicum JAZ3 (NP_001234373, XP_010317740), Solanum/ycopersicum JAZ1 (NP_001234883, ABU88421), Catharanthus roseus JAZ10 (ALI87033), Catharanthus roseus JAZ8 (ALI87032), Catharanthus roseus JAZ3 (ALI87031), Oryza sativa JAZ (BAT08274), Gossypium barbadense JAZ10 (AJT58397), Hevea brasiliensis JAZ (ADI39634), Gossypium barbadense JAZ2 (AJT58398), Hevea brasiliensis JAZ2 (AlY25007), Vitis rupestris JAZ2 (AEP60133), Vitis quinquangularis JAZ2 (AFJ23868.1), Artemisia annua JAZ2 (AJK93413), Pyrus pyrifolia JAZ1 (AGZ89627), Prunus persica JAZ1 (ADU76348), Nicotiana tabacum JAZ1 (ADZ48593), Genlisea aurea JAZ1 (EPS61226), Artemisa annua JAZ1 (AJK93412), Hevea brasiliensis JAZ11 (AlY25012), Eutrema halophilum JAZ12 (BAJ34102), Gossypium barbadense JAZ13 (AJT58403), Brassica napus JAZ (XP_013682524, XP_013682522, XP_013648884, XP_013648883), Brassica oleracea JAZ (XP_013590008, XP_013590007, XP_013618140), Gossypium raimondii JAZ (XP_012477278, XP_012477277), Gossypium arboreum JAZ (KHG27959), Brassica rapa JAZ (XP_009128069), Eucalyptus grandis (KCW79370, KCW79369, XP_010047457, Elaeis guineensis (XP_010939885, XP_010937308), Nelumbo nucifera (XP_010273865), Populus euphratica (XP_011039955), Glycine max TYFY 10A-like (XP_006587054, XP_003546514, NP_001276248), Glycine soja TIFY 10A-like (KHN12298, KHN07885), Vitis vinifera (CBI27776), Vitis vinifera TIFY 10A-like (XP_002277157, XP_002272363), Cicer arietinum TIFY 10A-like (XP_004488102), Lotus japonica (AFK35276), Ricinus communis (XP_002516243), Zea mays (NP_001182812, XP_002462448, NP_001130163), Malus domestica TIFY 10A-like (XP_008337828, XP_008388962), Phoenix dactylifera TIFY 10A-like (XP_008784340), Citrus clementina (XP_006452845), Jatropha curcas TIFY 10A-like (XP_012077082.1). In the list are included other sequences of the JAZ family from maize, rice, wheat, oilseed rape/canola, sorghum, soybean, sunflower, alfalfa, potato, tomato, tobacco, grape, barley, pea, bean, field bean, lettuce, cotton, sugar cane, sugar beet, broccoli or other vegetable brassicas or poplar among many others. In the list are also included other sequences of the JAZ family from ornamental plants, plants useful in timber (wood) production (such as pine tree, oak, chestnut or beech tree) and fruit trees (for example orange tree, pear tree, apple tree, olive tree, cherry tree or peach tree).

The nucleotide sequences of the present invention can be isolated from any plant, particulartly from any crop plant, but can be also from ornamental plants, timber plants and fruit plants. The sequences can be identified by means techniques known in the art (e.g. PCR) and isolated the entire sequence or fragments thereof.

A “fragment” of a polypeptide is included within the present invention if it retains substantially the same activity as the protein of the third aspect of the invention.

Herein the terms “amino acid sequence”, “polypeptide”, “peptide” and “oligopeptide” are used interchangeably.

A fourth aspect of the invention relates to a host cell comprising the isolated nucleic acid of the first aspect of the invention, or the vector of the second aspect of the invention, or the protein of the third aspect of the invention, with the proviso that when the host cell comprises the protein, the host cell is a plant cell but not an Arabidopsis thaliana cell, and specifically, the host cell is a guard cell of the stomata.

The cell can be named as “host cell”. The host cells as it is used in the present invention relates to any procariotic or eukaryotic cell or viruses that replicate in prokaryotic or eukaryotic cells. Essentially, it refers to a eukaryotic plant cell and within this group, more preferably, those cells belonging to the Kingdom Plantae, wherein any of these cells comprises the polynucleotide of the invention, or the vector of the invention. Thus, the term plant cell comprises at least one cell of the parenchyma, meristematic cell or of any kind of plant cell, differentiated or undifferentiated. Preferably the plant cell is the guard cell of the stomata, or the mesophyll cells.

To refer to any host cell comprising the polynucleotide of this invention, the term “host cell of the invention” or “host cell of the present invention” can be used.

Viral hosts for expression of the proteins of the present invention include viral particles, which replicate in prokaryotic host or viral particles, which infect and replicate in eukaryotic hosts. Procedures for generating a vector for delivering the isolated nucleic acid or a fragment thereof are well known. Suitable vectors include, but are not limited to, disarmed Agrobacterium tumor inducing (Ti) plasmids (e. g., pBIN19) containing a target gene under the control of a vector, such as the cauliflower mosaic (CaMV) 35S promoter or its endogenous promoter, tobacco mosaic virus and the like.

Once the vector has been prepared for expression, the nucleic acid may be introduced or transformed into an appropriate host. Various techniques may be employed for the transformation such as protoplast fusion, calcium phosphate precipitation, electroporation, or other conventional techniques. As is well known, viral sequences may be directly transformed into a susceptible host or first packaged into a viral particle and then introduced into a susceptible host by infection. After the cells have been transformed with the vector, or the virus or its genetic sequence is introduced into a susceptible host, the cells are grown in media and screened for appropriate activities. Expression of the sequence results in the production of the protein of the third aspect of the invention.

Numerous procedures are known in the art to assess whether a transgenic plant comprises the isolated nucleic acid of the first aspect of the invention or the vector of the second aspect of the invention, and need not be reiterated.

Cells which have stably integrated the nucleic acid of the present invention into their chromosomes can be selected by also introducing one or more reporter genes or markers which allow for selection of host cells which contain the expression vector. The reporter gene or marker may complement an auxotrophy in the host (such as leu2, or ura3, which are common yeast auxotrophic markers), biocide resistance, e.g., antibiotics, or resistance to heavy metals, such as copper, or the like. The selectable marker gene can either be directly linked to the DNA gene sequences to be expressed, or introduced into the same cell by co-transfection.

The present invention further relates to a cell culture comprising the host cell of the invention. To refer to any cell culture comprising the host cell of the present invention, the term “cell culture of the invention” or “cell culture of the present invention” can be used.

The term “cell culture” refers to a cultivation of cells isolated from the same or different type of tissue, or a collection of these cells organized in parts of a plant or in tissues (tissue culture). Types of this kind of cultures are, e.g. but without any limitation, a culture of protoplasts, calli (groups of plant cells undifferentiated able to regenerate a complete plant with the appropriate organogenic program) or a culture of plant cells that are isolated from plants or parts of plants such as embryos, meristematic cells, pollen, leaves or anthers.

A fifth aspect of the invention relates to a plant comprising the isolated nucleic acid of the first aspect of the invention, or the vector of the second aspect of the invention, or the protein of the third aspect of the invention; with the proviso that when the host cell of the plant comprises the protein the host cell is a guard cell of the stomata and wherein the plant is not Arabidopsis thaliana.

The plant of the fifth aspect of the present invention can be generated by transgenesis, genome editing technologies, truncation of its own Jas motif in the ZIM-domain containing proteins, chemical random mutagenesis and selection of the mutant, or by selection of T-DNA mutants. As new genome editing technologies, technologies such as ZFNs, TALENs or CRISPR/Cas (Boch, J. et al. Science. 2009; 326(5959): 1509-1512; Christian, M. et al. Genetics. 2010; 186(2): 757-761; Belhaj, K. et al. Plant Methods. 2013; 9(1): 39) could be used to artificially generate the nucleic acid of the present invention in crops such as maize, rice, wheat, oilseed rape/canola, sorghum, soybean, sunflower, alfalfa, potato, tomato, tobacco, grape, barley, pea, bean, field bean, lettuce, cotton, sugar cane, sugar beet, broccoli or other vegetable brassicas or poplar among many others for example, ornamental plants, plants useful in timber (wood) production (such as pine tree, oak, chestnut or beech tree) and fruit trees (for example orange tree, pear tree, apple tree, olive tree, cherry tree or peach tree). Preferably, the plant of the invention is a tomato plant.

By “transgenic plant” as used herein, is meant a plant, plant cell, tissue, flower, organ, including seeds, progeny and the like, or any part of a plant, which comprises an exogenously inserted gene or a targeted genomic modification to one or more alleles of an endogenous gene where the product of the gene produces a product that results in plant improved resistance to biotrophic pathogens without increasing susceptibility to necrotrophs. The manipulated gene herein is designated as “transgene”, independently of whether the gene has been introduced exogenously or the endogenous gene has been manipulated; in both cases, the sequence defined as “transgene” has not been shown to be naturally occurring.

The non-transgenic, wild-type, non-transgenic plants or wild type plants, as used herein, mean a plant without said genetic modification. These terms can refer to a cell or a part of a plant such as an organelle like a chloroplast or a tissue, in particular a plant, which lacks said genetic modification but is otherwise as identical as possible to the plants with at least one genetic modification employed in the present invention. The transgenic transcription product may also be oriented in an antisense direction.

The generation of transgenic plants comprising the isolated nucleic acid of the first aspect of the invention (in sense or antisense orientation) may be accomplished by transforming the plant with a plasmid, liposome, or other vector encoding the desired DNA sequence. Such vectors would, as described above, include, but are not limited to the disarmed Agrobacterium tumor-inducing (Ti) plasmids containing a sense or antisense strand. Methods of generating such constructs, plant transformation and plant regeneration methods are well known in the art once the sequence of the gene of interest is known.

The plant of the invention can be achieved by genetic transformation mediated by biolistic, Agrobacterium tumefaciens or any other technique known by the skilled in the art (e.g. transformation of protoplasts), that will allow the integration of the polynucleotide of the invention in any of the DNA of the plant; genomic, chloroplastic or the polynucleotide of the invention by crossing and selection.

The use of the CRISPR (Clustered Regularly Interspaced Short Palindromic Repeats)/Cas system to modify/edit the endogenous JAZ genes, preferably the endogenous JAZ2 gene, in any plant species requires the transformation of the plant with a suitable vector that expresses the CRISPR/Cas nuclease and the guiding RNA (gRNA) complementary to a part of the JAZ sequence, preferably JAZ2 sequence, as known by the expert in the field. Examples of this technique can be found in US2014273235A1.

The production of a plant can be performed by means of techniques known by the skilled in the art, for instance:
The cultivation of embryos: isolation of zygotic embryos promoting their growth as plant in an artificial environment
Somatic embryogenesis: production of embryos from somatic tissues, such as microspores or leaves
Organogenesis: production of organs such as stems or roots from various tissues of the plant.

The plant of the present invention can be obtained according with other microbiologic processes as for example:
Obtaining cybrids: it is produced a cell with its cytoplasm and the cytoplasm of the other cell and this cell can be grown in a suitable medium to produce a plant which expresses the heterologous polynucleotide.
Fusion of somatic cells (preferably protoplast fusion): at the cytoplasmic level hybrid plants can be the result of: (a) the sum of the cytoplasm of both parental; (b) the cytoplasm of a single parental; (c) a cytoplasm hybrid result of recombination of the genomes extranuclears of both cells. The fusion of somatic cells can be applied; to overcome the incompatibility in interspecific crosses, or a better utilization of the interspecies variation and extraspecific in interspecific compatible crosses.

The plant in the present invention can be any plant with stomata. Preferably the plant is a plant that can be infected by any biotroph pathogen through the stomata. Preferably the plant is any tree propagated or cultured by cuttings, such as fruit trees (for example orange tree, pear tree, apple tree, olive tree, cherry tree, apricot or peach tree); crops such as maize, rice, wheat, oilseed rape/canola, sorghum, soybean, sunflower, alfalfa, potato, tomato, tobacco, grape, barley, pea, bean, field bean, lettuce, cotton, sugar cane, sugar beet, broccoli or other vegetable brassicas or poplar beans cassava, cotton, cucurbits, pepper and cereals; ornamental plants; or plants useful in timber (wood) production (such as pine tree, oak, chestnut or beech tree), wherein the plant has resistance to biotrophs, preferably to biotrophic bacteria such as Pseudomonas syringae, Raistona, Erwinia and Xanthomonas species, and necrotrophs, preferably Botrytis cinerea, Fusarium, Plectosphaerella cucumerina and Alternaria brassicicola, among many others.

In accordance with the present invention, plants included within its scope include both higher and lower plants of the Plant Kingdom. Mature plants, including rosette stage plants, and seedlings are included in the scope of the invention. A mature plant, therefore, includes a plant at any stage in development beyond the seedling. A seedling is a very young, immature plant in the early stages of development.

Preferred plants of the present invention, but are not limited to, high yield crop species (including monocots and dicots, e. g., alfalfa, cashew, cotton, peanut, fava bean, french bean, mung bean, pea, walnut, maize, potato, sugar beet, tobacco, oats, wheat, barley and the like), or engineered endemic species. Particularly preferred plants are those from: the Family Umbelliferae, particularly of the genera Daucus (particularly the species carota, carrot) and Apium (particularly the species graveolens, celery) and the like; the Family Solanacea, particularly of the genus Solanum, particularly the species lycopersicum (tomato), tuberosum (potato) and melongena (eggplant or aubergine), and the like, and the genus Capsicum, particularly the species annuum (pepper) and the like; and the Family Leguminosae, particularly the genus Glycine, particularly the species max (soybean) and the like; and the Family Cruciferae, particularly of the genus Brassica, particularly the species rapa (turnip) and oleracea (red cabbage, cauliflower or broccoli) and the like; the Family Compositae, particularly the genus Lactuca, and the species sativa (lettuce), and the like.

By “plant” as used herein, is meant any plant and any part of such plant, be it wild-type, treated, genetically manipulated or recombinant, including transgenic plants. The term broadly refers to any and all parts of the plant, including plant cell, tissue, flower, leaf, stem, root, organ, and the like, and also including seeds, progeny and the like, whether such part is specifically named or not.

A preferred embodiment of this invention refers to any plant described above wherein the expression of the polynucleotide of the invention is greater than the expression of the homologous native gene JAZ2 under the control of promoters targeting the JAZ nucleic acid of the present invention to the stomata, for example using the promoter MYB60.

The plant contains the polynucleotide of the invention in homozygosis, heterozygosis or hemizygosis. As used herein the terms “Homozygous” or “Homozygosity” are understood within the scope of the invention and refer to a plant which is homozygous for a particular gene when identical alleles (forms of a given gene) of the gene are present on both homologs. As used herein, the term “homozygote” refers to an individual cell, plant or germplasm having the same alleles at one or more loci in homologous chromosomal segments. As used herein the terms “Heterozygous” or “Heterozygosity” are understood within the scope of the invention and refer to a plant which is heterozygous for a particular gene when two different alleles occupy the gene's position on the homologous chromosomes. As used herein, the term “heterozygote” refers to a diploid or polyploid individual cell, plant or germplasm having different alleles present at least at one locus. As used herein, the term “hemizygous” or “hemizygosis” refers to a genetic condition of a cell, tissue or organism where a gene or a nucleotic sequence is present in one of the homologous chromosomes, but is not found at the corresponding locus of the other homologous chromosome. For example, a plant which is said to be hemizygous at a given locus for a gene of interest or for a certain sequence only contains the said transgene or the said sequence at that locus of one but not the other one of the respective pair of homologous chromosomes.

A sixth aspect of the present invention refers to a germplasm of the plant of the fifth aspect of the invention. Additionally, the germplasm of the present invention comprising the isolated nucleic acid, the protein, or the host cell of the invention with the proviso that the germplasm is not an A. thaliana germplasm.

The germplasm is defined by the biological material that contains the intraspecific genetic variability or by the genetic materials that can perpetuate a species or a population of said plant. Particularly, germplasm refers to a genetic material of or from an individual (e.g., a plant), a group of individuals (e.g., a plant line, variety or family), or a clone derived from a line, variety, species, or culture. The germplasm can be part of an organism or cell, or can be separate from the organism or cell. In general, germplasm provides genetic material with a specific molecular makeup that provides a physical foundation for some or all of the hereditary qualities of an organism or cell culture. Thus germplasm includes cells, seed, tissue culture for any part of the plant from which new plants may be grown, or plant parts, such as leafs, stems, pollen, seed, or cells that can be cultured into a whole plant, or plants established in ex situ collections, without excluding any other material in the scope of this definition.

The pollen has high level of interest since the transmission of the genetic and phenotypic characters can be carried out by the pollination of any plant variety compatible with the pollen that is referenced. In this way can be produce a plant which includes the polynucleotide of the invention, after the respective cross and selection, it can be obtained a plant in which the polynucleotide integrates a suitable number of copies in stable condition in order to obtain the same desirable phenotype in the subsequent generations.

A preferred embodiment of the present invention refers to a plant comprising the isolated nucleic acid of the first aspect of the invention, wherein said plant is obtainable by introgression from the plant of the fifth aspect of the invention, or from the germplasm of the sixth aspect of the invention, and preferably the plants are inbred or hybrid plants.

The present invention also refers to a plant obtainable by means the introgression of the modified JAZ nucleic acid from the plant of the fifth aspect of the invention, or from the germplasm of the sixth aspect of the invention in any plant having a promoter that regulates the specific expression of the nucleic acid in the guard cells of the stomata, with the proviso that the modified JAZ nucleic acid of the obtained plant was operably linked to said promoter that regulates the specific expression of the nucleic acid in the guard cells of the stomata, and preferably the plants are inbred or hybrid plants.

A seventh aspect of the present invention refers to a use of the isolated nucleic acid of the first aspect of the invention, or the vector of the second aspect of the invention, or the protein of the third aspect of the invention, or the cell of the fourth aspect of the invention to produce a plant with resistance to biotrophic or hemi-biotrophic plant pathogens, preferably Pseudomonas syringae (for example pathovars tomato or maculicola), and without modifying the level of susceptibility of said plant to necrotrophic plant pathogens, preferably Botrytis cynerea. The term “hemi-biotrophic” is well known in the art. The term “without modifying the level of susceptibility” as used in the present invention means that the tolerance and/or resistance is not substantially altered.

In the present invention among biotrophic or hemi-biotrophic plant pathogens the following can be found: examples of biotrophic or hemi-biotrophic pathogens include Pseudomonas sp (for example Pseudomonas solanacearum and Pseudomonas syringae, specifically pathovars tomato and maculicola), Xanthomonas sp (Xanthomonas campestris, specifically pathovars campestris and vesicatoria), Hyaloperonospora sp, Erisyphe sp, Ralstonia sp, Erwinia sp and Magnaporthe sp. Many other such examples are well known in the art.

In the present invention among necrotrophic plant pathogens the following can be found: Botrytis cinerea, Fusarium sp., Alternaria brassicicola, Plectosphaerella cucumerina, Phytophthora infestans, Peronospora parasitica, Rhizoctonia solani, Phoma lingam (Leptosphaeria maculans), and Albugo candida. Many other such examples are well known in the art.

An eight aspect of the present invention refers to a method for producing a plant with resistance to biotrophic or hemi-biotrophic plant pathogens, preferably to Pseudomonas syringae, and without modifying the level of susceptibility of said plant to necrotrophic plant pathogens, preferably Botrytis cinerea, comprising:

a. transferring to the isolated plant material the isolated nucleic acid sequence of the first aspect of the invention, wherein said transfer of said nucleic acid is performed by transformation, by protoplast fusion, by a doubled haploid technique, by gene gun, by electroporation, by viral transduction, or by embryo rescue, provided that when said method involves a doubled haploid technique said method is not essentially biological;

b. identifying the plant material obtained in the step (a) comprising the modified JAZ nucleic acid operably linked to a promoter that regulates the specific expression of the nucleic acid in the guard cells of the stomata, as defined in the first aspect of the invention;

c. growing the plant material identified in the step (b) in a suitable medium to produce at least a plant and/or a germplasm which expresses the modified JAZ nucleic acid sequence.

A ninth aspect of the present invention refers to a method for producing a plant with resistance to biotrophic or hemi-biotrophic plant pathogens, preferably to Pseudomonas syringae, and without modifying the level of susceptibility of said plant to necrotrophic plant pathogens, preferably Botrytis cinerea, comprising:

a. modifying the JAZ native nucleic acid of the isolated plant material in order to obtain the nucleic acid encoding the Jas domain as defined in the nucleic acid of the first aspect of the invention by means of the Zinc finger nuclease 1 and 2 (ZFN1 and 2) technology, TALENS or a CRISP/Cas technology;

b. identifying the plant material obtained in the step (a) having the nucleic acid encoding a non-functional Jas domain,

c. growing the plant material identified in the step (b) in a suitable medium to produce at least a plant and/or a germplasm which expresses the modified JAZ nucleic acid sequence.

A tenth aspect of the present invention refers to a plant obtained by a method of the eight or the ninth aspect but not A. thaliana plant. Preferably, the plant of the invention is a tomato plant.

An eleventh aspect of the present invention refers to a method for detecting a plant with resistance to biotrophic or hemi-biotrophic plant pathogens, preferably to Pseudomonas syringae, and without modifying the level of susceptibility of said plant to necrotrophic plant pathogens, preferably Botrytis cinerea, that comprises detecting the modified JAZ nucleic acid operably linked to a promoter that regulates the specific expression of the nucleic acid in the guard cells of the stomata, as defined in the first aspect of the invention.

The detection of the modified JAZ nucleic acid operably linked to a promoter that regulates the specific expression of the nucleic acid in the guard cells of the stomata can be carried out by means techniques known in the art (e.g. PCR).

The present invention also refers to a kit that comprises the nucleic acid sequence of the first, the vector of the second aspect of the present invention, or the protein of the third aspect of the present invention or the cell of the fourth aspect of the present invention artificially introduced by using the new genome editing technologies, such as ZFNs, TALENs or CRISPR/Cas in selected crops with appropriate vectors. The kit can also comprise sequences to express the CRISPR/Cas nuclease and/or the sequence for the guiding RNA that is complementary to a part of the modified JAZ sequence. Also the kit can comprise the means to use the ZFNs and/or TALENs technology.

The present invention also refers to the use of the kit of the invention for the production of a plant with resistance to biotrophs, preferably to biotrophic or hemi-biotrophic plant pathogens, preferably to Pseudomonas syringae, Raistonia, Erwinia and Xanthomonas species, and without modifying the level of susceptibility of said plant to necrotrophic plant pathogens, preferably Botrytis cinerea, Fusarium sp. and Alternaria brassicicola, among many others.

Unless otherwise defined, all technical and scientific terms used herein have the same meaning as commonly understood by one of ordinary skilled in the art to which this invention belongs. Methods and materials similar or equivalent to those described herein can be used in the practice of the present invention. Throughout the description and claims the word “comprise” and its variations are not intended to exclude other technical features, additives, components, or steps. Additional objects, advantages and features of the invention will become apparent to those skilled in the art upon examination of the description or may be learned by practice of the invention. The following examples, drawings and sequence listing are provided by way of illustration and are not intended to be limiting of the present invention.

### BRIEF DESCRIPTION OF THE DRAWINGS

FIG. 1. Tissue Expression Patterns of JAZ2.

Histochemical GUS activity of 6 days old Arabidopsis transgenic seedlings expressing the GUS reporter gene under the control of the JAZ2 promoter (pJAZ2:GUS). Seedlings were treated with 50 μM of JA or mock for 2 hours. GUS activity was detected after overnight staining.

FIG. 2. Isolation of the Arabidopsis JAZ2-3 mutant.

(A) JAZ2 gene model showing JAZ2-3 transposon insertion sites. (B) RT-PCR to detect full-length JAZ2 transcript accumulation in WT and JAZ2-3 mutant after treatment with 1 μM COR (+) or a mock solution (−). (C) Quantitative RT-PCR to detect JAZ2 transcript accumulation in WT and JAZ2-3 plants after treatment with 1 μM COR or a mock solution using primers that amplify an AtJAZ2 C-terminal fragment located after the insertion.

FIG. 3. JAZ2 regulates COR-induced stomatal dynamics during Pseudomonas infection

(A) Stomatal aperture in Arabidopsis No-0, No-0 JAZ2-3, Col-0 and Col-0 coi1-30 leaves measured after 5 hours of incubation with OD₆₀₀=2 of MAMPs, OD₆₀₀=2 of MAMPs and 2 μM of COR or a mock control. Error bars indicate standard error of the mean (SEM) (n=20). Different letters above columns indicate significant differences compared with mock-treated No-0 control sample (Student's t test, p<0.001). The results are representative of four independent experiments. (B) Quantitative RT-PCR analysis of PR1 and PR2 expression on Arabidopsis No-0 and JAZ2.3 seedlings induced for 5 or 20 hours with MAMPs (OD₆₀₀=1.5 of boiled Pto DC3000) or a mock control. The measurements (three technical replicates) represent the ratio of expression levels between each sample and mock treated No-0 at each time point. All samples were normalized against the housekeeping gene AtACT8. Error bars represent standard deviation (SD). The results are representative of two independent experiments. Asterisks indicate statistically significant differences in the gene induction between MAMP induced Arabidopsis No-0 and JAZ2.3 lines as indicated (Student's t test, *p<0.05; **p<0.01; ***p<0.001). (C) Growth of Pto DC3000 on Arabidopsis No-0 and No-0 JAZ2-3 plants three days after spray inoculation with bacteria at 10⁸ colony-forming units mL⁻¹ (cfu/ml) or syringe infiltration with bacteria at 5×10⁵ cfu/ml. Error bars indicate SEM (n=7). Asterisks indicate statistically significant values in J.4Z2-3 lines compared to its No-0 WT control (Student's t test, *p<0.05; **p<0.01; ***p<0.001). The results are representative of three independent experiments

FIG. 4. Isolation of the JAZZ2ΔJas and JAZ1ΔJas mutants.

(A) JAZ2 gene model showing the T-DNA insertion site in JAZ2ΔJas. The Jas motif coding region is shown in dark grey. (B) RT-PCRs to detect JAZ2 transcript accumulation in WT (Col-0) and JAZ2ΔJas Arabidopsis mutants after treatment with 50 μM JA (+) or a mock solution (−) for 2 hours. Three different regions of JAZ2 were amplified to characterize the JAZ2ΔJas Arabidopsis mutant: an AtJAZ2 full-length fragment, an AtJAZ2 N-terminal fragment located before the insertion and finally, an AtJAZ2 C-terminal fragment located after the insertion. (C) JAZ1 gene model showing the T-DNA insertion in JAZ1ΔJas. The Jas motif coding region is shown in dark grey.

FIG. 5. Tissue Expression Patterns of JAZ1, and JAZ3.

Histochemical GUS activity of 6 days old Arabidopsis transgenic seedlings expressing the GUS reporter gene under the control of the promoter of JAZ1 (pJAZ1:GUS) (A) or JAZ3 (pJAZ3:GUS) (B). For treatment with the hormone (JA panels), the seedlings were treated with 50 μM of JA for 2 hours. GUS activity was detected after overnight staining.

FIG. 6. Dominant JAZZ2ΔJas mutants are impaired in COR-induced stomata reopening and resistance to P. syringae infections.

(A) Stomatal aperture in Arabidopsis Col-7, JAZ1ΔJas, Col-0, JAZ2ΔJas, JAZ3ΔJas and coi1-30 leaves measured after 5 hours of incubation with MAMPs (OD₆₀₀=1.5 of crude Pto DC3000 extracts), MAMPs plus 2 μM COR or a mock control. Error bars indicate SEM (n=20). Different letters above columns indicate significant differences compared to their respective mock-treated VVT control sample (Col-7 or Col-0) (Student's t test, p<0.001). The results are representative of three independent experiments.

(B) Pto DC3000 disease symptoms on Col-7, JAZ1ΔJas, Col-0, JAZ2ΔJas, JAZ3ΔJas and coi1-30 plants after spray inoculation with Pto DC3000 bacteria at 10⁸ cfu/ml. Pictures were taken 4 days after inoculation and show typical chlorosis caused by Pto DC3000. Leaves show representative symptoms of three independent experiments.

(C) Growth of Pto DC3000 on Arabidopsis Col-7, JAZ1ΔJas, Col-0, JAZ2ΔJas, JAZ3ΔJas and coi1-30 plants three days after spray inoculation with bacteria at 10⁸ cfu/ml or syringe infiltration with bacteria at 5×10⁵ cfu/ml. Error bars indicate SEM (n=7). Different letters above columns indicate significant differences compared to their respective WT (Col-7 or Col-0) control samples in each infection condition (Student's t test, p<0.05). ns: no significant. The results are representative of three independent experiments.

(D) Growth of Pto DC3000 or a COR-deficient Pto DC3000 strain (Pto DC3000 cor-) on Arabidopsis Col-0, JAZ2ΔJas and coi1-30 plants three days after spray inoculation with bacteria at 10⁸ cfu/ml. Error bars indicate SEM (n=7). Different letters above columns indicate significant differences compared to WT plants in each infection condition (Student's t test, p<0.001). The results are representative of three independent experiments.

FIG. 7. COR induced degradation of JAZ2-GUS at stomata.

Histochemical GUS activity of 8 days old Arabidopsis transgenic seedlings expressing the GUS reporter gene or JAZ2-GUS under the control of the 35S promoter. Seedlings were treated with 50 nM of COR or a mock solution for 3 hours. GUS activity was detected after an overnight staining.

FIG. 8. Effect of the JAZZ2ΔJas mutation on root growth inhibition and anthocyanin accumulation by COR.

(A) and (B) Root growth inhibition assays of 11 days old Arabidopsis seedlings from Col-7, JAZ1ΔJas, Col-0, JAZ2ΔJas, JAZ3ΔJas, No-0, No-0 JAZ2-3 and coi1-30 plants grown in 1 μm COR or mock media. Results shown are the mean ±SD of measurements from 30 seedlings. (C) Anthocyanin accumulation of 10 days old Arabidopsis seedlings from Col-7, JAZ1ΔJas, Col-0, JAZ2ΔJas, JAZ3ΔJas, No-0, No-0 JAZ2-3 and coi1-30 plants grown in 1 μM COR or mock media. Results shown are the mean ±SD of measurements from 30 seedlings.

FIG. 9. MYC2, MYC3 and MYC4 are expressed at stomata and control COR-induced stomatal reopening

(A) MYC3 and MYC4 expression patterns at stomata guard cells. Data from Arabidopsis eFP Browser, http://bbc.botany.utoronto.ca/efp/cgi-bin/efpWeb.cgi (B) Histochemical GUS staining of MYC3 and MYC4 expression at stomata guard cells, (C) Stomatal aperture in Arabidopsis Col-0, myc2, myc2myc3, Myc2myc3myc4 and coi1-30 leaves measured after 5 hours of incubation with MAMPs (OD₆₀₀=1.5 of crude Pto DC3000 extracts), MAMPs plus 2 μM COR or a mock control. Error bars indicate SEM (n=20). Different letters above columns indicate significant differences compared to mock-treated Col-0 control sample (Students t test, p<0.1). The results are representative of three independent experiments. (D) Growth Pto DC3000 on Arabidopsis Col-0, myc2, myc2myc3, myc2myc3myc4 and coi1-30 plants three days after spray inoculation with bacteria at 10⁸ cfu/ml or syringe infiltration with bacteria at 5×10⁵ cfu/mL Error bars indicate SEM (n=7). Asterisks indicate statistically significant between the indicated Arabidopsis lines (Students t test, *p<0.05; **p<0.01; ***p<0.001). ns: no significant. The results are representative of two independent experiments. (E) Growth of Pto DC3000 or a COR-deficient Pto DC3000 strain (Pto DC3000 cor-) on Arabidopsis Col-0 and myc2myc3myc4 plants three days after spray inoculation with bacteria at 10⁸ cfu/ml. Error bars indicate SEM (n=7). Asterisks indicate statistically significant differences compared to WT plants in each infection condition at *p<0.05; **p<0.01; ***p<0.001). The results are representative of two independent experiments.

FIG. 10. MYC2, MYC3, MYC4 and JAZ2 are required for full COR-induction of ANAC19, ANAC55 and ANAC72 expression

(A) Quantitative RT-PCR analysis of ANAC19, ANAC55 and ANAC72 expression on Arabidopsis Col-0, myc2, myc2myc3, myc2myc3myc4 and coi1-30 seedlings induced for 24 hours with 1 μM of COR or a mock solution. The measurements (three technical replicates) represent the ratio of expression levels between each sample and mock treated Col-0. All samples were normalized against the housekeeping gene AtACT8. Error bars represent standard deviation (SD). The results are representative of two independent experiments. Asterisks indicate statistically significant differences in the gene induction in each Arabidopsis line compared to the previous (Student's t test, **p<0.01). ns: no significant.

FIG. 11. MYC2, MYC3, MYC4 and JAZ2 are required for full COR-induction of ANAC19, ANAC55 and ANAC72 expression

Quantitative RT-PCR analysis of ANAC19, ANAC55 and ANAC72 expression on Arabidopsis Col-0, myc2, myc2myc3, myc2myc3myc4, JAZ2ΔJas and coi1-30 seedlings induced for 20 hours with 1 μM of COR or a mock solution. The measurements (three technical replicates) represent the ratio of expression levels between each sample and mock treated Col-0. All samples were normalized against the housekeeping gene AtACT8. Error bars represent standard deviation (SD). The results are representative of three biologically independent experiments. Asterisks indicate statistically significant differences in the gene induction in each Arabidopsis line compared to the previous as indicated (Student's t test, *p<0.05; **p<0.01; ***p<0.001). ns: no significant.

FIG. 12. MYC2 and MYC3 directly bind to the promoter of ANAC19, ANAC55 and ANAC72 genes.

MYC2-YPet and MYC3-YPet transgenic plants under the control of their native promoters were used for ChlP-PCR experiments. Graphic shows fold enrichment of Q-PCR data from Chlp assays with antibody against GFP, which cross-reacts with the GFP derivative YPet, using the ACTIN8 gene as negative control. Error bars represent SD of two technical replicates. Each experiment was repeated twice with similar results. Asterisks indicate statistically significant differences compared to Col-0 for each gene (Student's t test, *p<0.05; **p<0.01; ***p<0.001).

FIG. 13. JAZ2ΔJas mutants retain WT resistance to necrotrophic fungi.

(A) B. cinerea disease symptoms (above) and determination of spore number (below) on Col-7, JAZ1ΔJas, Col-0, JAZ2ΔJas, JAZ3ΔJas, JAZ10ΔJas and coi1-30 plants 6 days after inoculation with 5×10⁶ spores per ml. (B) P. cucumerina disease symptoms and determination of spore number on Col-0, JAZ2ΔJas and coi1-30 plants 6 days after inoculation with 3×10⁷ spores per ml. (C) A. brassicicola disease symptoms and determination of spore number on Col-0, JAZ2ΔJas and coi1-30 plants 6 days after inoculation with 1×10⁶ spores per ml. Leaves in A, B and C show representative symptoms of at least two independent experiments. For quantification, three pools per treatment (containing five leaves from five independent plants per pool) were measured. Error bars in A, B and C represent standard deviation (SD). Asterisks indicate statistically significant differences with their respective VVT controls (Col-0 or Col-7) (Student's t test, *p<0.05; **p<0.01; ***p<0.001).

FIG. 14. SIJAZ1 and SIJAZ2 are phylogenetically closest to AtJAZ2. Phylogenetic tree of JAZs from tomato (SIJAZ) and Arabidopsis (AtJAZ). AtJAZ2 is more similar to SIJAZ1 and SIJAZ2 than to the other remaining ten SIJAZs.

FIG. 15. SIJAZ2 is more expressed (enriched) at stomata compared to mesophyll cells. (A) Gene expression analyses of different tomato JAZs (SIJAZ1-4) in whole leaves. (B) Gene expression analyses of different tomato JAZs (SIJAZ1-4) in epidermal peelings (enriched with stomata guard cells). Expression data in A and B are relative to SIJAZ4. (C) Ratio of SIJAZ expression for each gene in stomata vs mesophyll cells.

FIG. 16. Schematic representation of SIJAZ2 mutants obtained by CRISPR/Cas9 technology. gRNA shows the sequence of the guide RNA used. Two independent lines are shown with two different deletions disrupting the functional JAS domain. Bold letters indicate the Jas domain.

FIG. 17. SIJAZ2Δjas mutants are more resistant to P. syringae pv. tomato DC3000 infection. Pictures of symptoms (A, B) or bacterial quantification (C) of wild type (WT) and homozygous CRISPR/Cas SIJAZ2ΔJas plants 7 days post infection with Pseudomonas DC3000. * p-value Tukey HSD <0.05.

FIG. 18. SIJAZ2ΔJas mutants retain WT resistance levels to Botrytis cinerea. Symptoms of tomato leaves (A) and quantification of lesion area (B) 4 days post infection (dpi) with Botrytis cinerea. Error bars indicate standard error (SE).

### EXAMPLES

### Material and Methods

Plant Growth Conditions

For in vitro assays, seedlings of six to eleven days were growth in MS or Johnson's medium at 21° C. under a 16-h-light/8-h-dark cycle. For stomata analysis and bacterial growth assays, A. thallana plants of four to six weeks were grown in controlled environment chambers at an average temperature of 22° C. (range 16° C.-24° C.), with 45%-65% relative humidity under short day conditions (8 h light).

Transgenic Arabidopsis lines expressing pJAZ:GUS promoter fusions were obtained by cloning the corresponding pJAZ PCR products (pJAZ1 fw caccGTACGTTCCAACTTCTAACC [SEQ ID NO: 29]; pJAZ1 rev CATCTTTAACAATTAAAACTTTCAAACG [SEQ ID NO: 30]; pJAZ2 fw caccGACTAAGAATTTGTTATGAAG [SEQ ID NO: 31]; pJAZ2 rev CATCGTTGAAACCGAAATTGAAATCG [SEQ ID NO: 32]; pJAZ3 fw caccGATCTGGCTCGACGTCGACGAGAAGC [SEQ ID NO: 33]; pJAZ3 rev CATCTATAATAAAGACACAGCCCGC [SEQ ID NO: 34]) into the pENTR™/D-TOPO® system (Invitrogen) and then transferring them to pGWB3. Agrobacterium strain GV3101, containing these constructs, was used to transform Col-0 plants by floral dipping (Clough, S. J. and Bent, A. F. Plant J. 1998, 16(6): 735-743). Several transgenic lines were checked for Kanamycin and Hygromycin resistance and lines carrying one insertion were driven to homozygosis and used for further analysis. pJAZ2:GUS in coi1-30 background was generated by crossing the corresponding parental single homozygous lines. Arabidopsis lines expressing pMYC3:GUS and pMYC4:GUS promoter fusions, were previously reported (Fernandez-Calvo, P. et al. Plant Cell. 2011, 23(2): 701-715). The lines JAZ2-3 (RIKEN_ 13-5433-1) and JAZ2ΔJas (GABI_169B06) were obtained from the Nottingham Arabidopsis Stock Centre. The line JAZ1ΔJas (SEQ ID NO: 61) was obtained from an Arabidopsis activation tagging population (Weigel, D. et al. Plant Physiol. 2000, 122(4): 1003-1013). Knock out lines myc2/jin1-2 (Lorenzo, O. et al., Plant Cell. 2004, 16(7): 1938-1950; Fernandez-Calvo, P. et al. Plant Cell. 2011; 23(2): 701-715), myc2/myc3, myc2/3/4 (Fernandez-Calvo, P. et al. Plant Cell. 2011; 23(2): 701-715), jai3-1 (Chini, A. et al. Nature. 2007; 448(7154)) and coi1-30 (Yang, D. L. et al. Proc Natl Acad Sci USA. 2012, 109(19): E1192-1200) were previously described. coi1-1 was provided by J. Turner.

Transgenic Arabidopsis plants expressing epitope-tagged MYC2 (AT1G32640) and MYC3 (AT5G46760) under control of their native promoters were generated by recombineering. A YPet-3xHA tag was fused to MYC2 and a YPet-6xHis-3xFLAG tag fused to MYC3, immediately before the stop codon and in-frame in both cases. YPet is an enhanced fluorescent tag derived from GFP. Fusions were generated in the transformation competent bacterial artificial chromosome (TAC) clones JAtY76E19 and JAtY69C18, respectively (Alonso, J. M. and Stepanova, A. N. Methods Mol Biol. 2015, 1227: 233-243). These constructs were transformed into wild-type Arabidopsis using floral dip methodology dipping (Clough, S. J. and Bent, A. F. Plant J. 1998, 16(6): 735-743). The expression of tagged MYC2 and MYC3 was confirmed by immunoblotting using an anti-GFP antibody that recognizes YPet. Homozygous transgenic lines exhibiting patterns of resistance marker gene segregation consistent with single site insertion were identified by selection of T2 and T3 generation seedlings on Linsmaier and Skoog medium plates (Caisson labs, USA) containing 15 μg/ml glufosinate ammonium (Sigma-Aldrich, USA). These lines were designated pMYC2:MYC2-YPet and pMYC3:MYC3-YPet, and used to perform ChlP-PCR experiments.

### GUS Staining

For JAZ promoter:GUS expression experiments, five to seven days-old seedlings were treated with mock solution or 50 μM JA for 2 h and GUS stained as previously described (Fernandez-Calvo, P. et al. Plant Cell. 2011; 23(2): 701-715). For JAZ2 protein stability experiments, seven to nine days-old seedlings were treated with mock solution or 50 nM COR for 3 h. Then, samples were placed in staining solution containing 50 mM phosphate buffer, pH 7, 0.1% (v/v) Triton X-100 (Sigma-Aldrich), 2 mM 5-bromo-4-chloro-3-indolyl b-D glucuronic acid (X-Gluc, Glycosynth), 1 mM potassium-ferrocyanide (Sigma-Aldrich), and 1 mM potassium-ferricyanide (Sigma-Aldrich) and incubated at 37° C. overnight. After staining, the tissue was soaked several times in 75% ethanol and kept in 5% glycerol until being photographed with a Leica DMR UV/VIS microscope.

### Bacterial Strain and Bacterial Growth Curves

Pseudomonas strains used in this study were Pseudomonas syringae pv. tomato (Pto) DC3000 and the coronatine-deficient Pto DC3000 strain (Pto DC3000 COR-) which is a Pto DC3000 AK87 mutant that carries mutations in cmaA (coronamic add A) and cfa6 (coronafacic acid 6).

Bacterial growth assays in Arabidopsis were performed as previously described (Gimenez-Ibanez, S. et al. Plant Signal Behay. 2009, 4(6): 539-541). For spraying infection assays, plants were sprayed with a bacterial suspension containing 10⁸ (cfu)/ml bacteria (OD₆₀₀=0.2) with 0.04% Silwet L-77. For bacterial growth assays by syringe infiltration, leaves were syringe infiltrated with a bacterial suspension containing 5×10⁵ (cfu)/ml bacteria (OD₆₀₀=0.001). Bacterial growth assays by spray inoculation and syringe infiltration were performed simultaneously and thus, results shown represent comparable experiments. Error bars indicate standard error of the mean (SEM) (n=7). These experiments were repeated at least three tires with similar results, and representative results are shown.

Stomatal Aperture Measurements

Whole leaves from 4- to 6-week-old Arabidopsis plants were collected and exposed to white light for 1.30 hours while floating in a solution containing 50 mM KCl, 10 μm CaCl₂, and 10 mm MES-KOH pH=6.1 (opening stomata buffer) with moderate shaking (50 rpm) to induce homogenous stomatal aperture among all Arabidopsis lines to compare. To do the MAMP-induced closure assays, the previous solution was removed and whole leaves were immersed in opening stomata buffer containing Pto DC3000 MAMP extracts at OD₆₀₀=1,5 (7,5×10⁸ cfu/ml), 2 μM of coronatine or a mock solution. Samples were further incubated under the same previous conditions for 4-5 hours. Then, leaves were rapidly peeled and abaxial epidermal peels were placed on cover slips and observed with a microscope (Leica DMR). Stomatal aperture (width/length) was measured using ImageJ software. Error bars represent standard error of the mean (SEM) (n=20). Stomatal experiments were repeated at least three independent times and representative results are shown.

Quantitative RT-PCR

Gene expression experiments were performed with RNA extracted from 7 to 10-day-old seedlings grown on liquid MS media that were treated with 1 μM of COR for 5 or 20 h or a mock solution. For experiments monitoring PR gene expression, seedlings were treated with DC3000 MAMP extracts at OD₆₀₀=1,5 (7,5×10⁸ cfu/ml), 2 μM of coronatine or a mock solution for 5 hours as stomata experiments were performed or 20 hours. Quantitative RT-PCR was performed as it was previously described (Fernandez-Calvo. P. et al. Plant Cell. 2011; 23(2): 701-715). Data analysis shown was done using three technical replicates from one biological sample; similar results were obtained with at least three additional independent biological replicates.

Primers for ANAC19, ANAC55 and ANAC72 genes used here were previously described (Zheng, X. Y. et al. Cell Host Microbe. 2012, 11(6): 587-596), and the complete list of primers used in this study are as follows:

AtANAC19fw:
[SEQ ID NO: 35]
5′-GCATCTCGTCGCTCAG-3′;
and
AtANAC19_rev
[SEQ ID NO: 36]
5′-CTCGACTTCCTCCTCCG-3′;;
AtANAC55_fw:
[SEQ ID NO: 37]
5′-GCGCTGCCTCATAGTC-3′
and
AtANAC55_rev
[SEQ ID NO: 38]
5′-CGAGGAATCCCCTCAGT-3′;
AtANAC72_hv:
[SEQ ID NO: 39]
5′-TGGGTGTTGTGTCGAAT-3′
and
AtANAC72_rev
[SEQ ID NO: 40]
5′-ATCGTAACCACCGTAACT-3′;
AtACTIN8_fw:
[SEQ ID NO: 41]
5′-CCAGTGGTCGTACAACCGGTA-3′
and
AtACTIN8_rev:
[SEQ ID NO: 42]
5′-TAGTTCTTTTCGATGGAGGAGCTG-3′;
AtJAZ2 Full Length Forward (AtJAZ2 FL_F),
[SEQ ID NO: 43]
5′-CGAGTGTTGGGACTTCTCTG-3′;
AtJAZ2 Full Length Reverse (AtJAZ2 FL_R),
[SEQ ID NO: 44]
5′-GCTCTTCTTGCAATCGGGAGT-3′;
AtJAZ2-3 Forward (AtJAZ2-3_F),
[SEQ ID NO: 45]
5′-TGGATTCTTCTGCTGGTCAA-3′;
AtJAZ2ΔJas Forward (JAZ2ΔJas F),
[SEQ ID NO: 46]
5′-TGCTTCTAGCCCAAATCCTG-3′;
AtJAZ2ΔJas Reverse (jAZ2ΔJas_R),
[SEQ ID NO: 47]
5′-TCTTTGGTCCCAGAGGAAGA-3′;
AtPR1,
[SEQ ID NO: 48]
5′-GTGGGTTAGCGAGAAGGCTA-3′
and
[SEQ ID NO: 49]
5′-ACTTTGGCACATCCGAGTCT-3′;
AtPR2,
[SEQ ID NO: 50]
5′-GTCTGAATCAAGGAGCTTAGCC-3′
and
[SEQ ID NO: 51]
5′-CCGTAGCATACTCCGATTTGT-3′;

Root Growth and Anthocyanin Measurements

Root growth, chlorophyll and anthocyanin measurements were performed as previously described (Fonseca, S. et al. PLoS One. 2014, 9(1): e86182.). In both cases, 20 to 30 seedlings were measured seven to eleven days after germination in presence or absence of 1 μm of COR or a mock control. Values represent mean and standard deviation (SD). Results are representative of at least two independent experiments.

ChlP-PCR

For pMYC2:MYC2-YPet and pMYC3:MYC3-YPet ChlP, ten-days-old seedlings were treated with1 μM of COR for 4 h. ChlP was performed as previously described (Busch, W. et al. Dev Cell. 2010, 18(5): 849-861) using an anti-GFP that cross-reacts with YPet and the samples were analyzed by qPCR. Input samples were first used to normalize the results. Fold difference was then calculated by taking ratios between normalized results from the probes (ChlP with antibody against GFP) and the corresponding control (ChlP with no antibody). Finally, the fold enrichment was calculated as the ratio between non-transgenic Col-0 plants and from pMYC2:MYC2-YPet or pMYC3:MYC3-YPet plants.

Primers were designed to amplify positions of putative MYC2 and MYC3 binding sites in ANAC019, ANAC055 and ANAC072 promoters at −991, −625 and −776 base pairs from transcription start site were, respectively. The primers used for qPCR are as follows:

AtANAC19 G-box (At ANAC19 G-box F),
[SEQ ID NO: 52]
5′-AGCAGCTACTACGAGTTGTGT-3′;
AtANAC19 G-box (At ANAC19 G-box R),
[SEQ ID NO: 53]
5′-CGTGTCCACGTGTCTATCGT-3′;
AtANAC55 G-box (At ANAC55 G-box F),
[SEQ ID NO: 54]
5′-TGTGTCGGCTTGTGGTAGTT-3′;
AtANAC55 G-box (At ANAC55 G-box R),
[SEQ ID NO: 55]
5′-GGGATGAGTTCACTGGATGGT-3′;
AtANAC72 G-box (At ANAC72 G-box F),
[SEQ ID NO: 56]
5′-TGCAATCACTCAGCGGACTT-3′;
AtANAC72 G-box (At ANAC72 G-box R),
[SEQ ID NO: 57]
5′-GGCCGACCTTATCGATGTGT-3′;
AtAC71N8 ChiP,
(AtACTIN8 ChIP F)
[SEQ ID NO: 58]
5′-GACTCAGATCATGTTTGAGACCTTT-3′
and
(AtACTIN8 ChIP R)
[SEQ ID NO: 59]
5′-ACCGGTTGTACGACCACTGG-3′.

Botrytis Cinerea Infection Assays

B. cinerea infection assays were performed as previously described (Monte, I. et al. Nat Chem Biol. 2014, 10(8): 671-676). Briefly five-week-old Arabidopsis plants were inoculated with 20 μl of a suspension of 5×10⁶ sporesiml PDB (Difco). At least 15 leaves (3 leaves per plant) were inoculated per treatment. Disease symptoms were scored 6 days after inoculation. Spores were quantified in a hemocytometer with a light microscope (Leica DMR UV/VIS). Five inoculated leaves of five different plants were pooled for each replicate. Three independent replicates were measured for each treatment. This experiment was repeated twice with similar results.

P. cucumerina and A. brassicicola infections assays were performed as previously described for B. cinerea but inoculating each leaf with 20 μl of a suspension of 3×10⁷ (P. cucumerina) or 1×10⁶ (A. brassicicola) sporestml PDB. Disease symptoms and spores were quantified as previously described for B. cinerea

### Example 1

### JAZ2 Gene is Expressed in Stomata Guard Cells

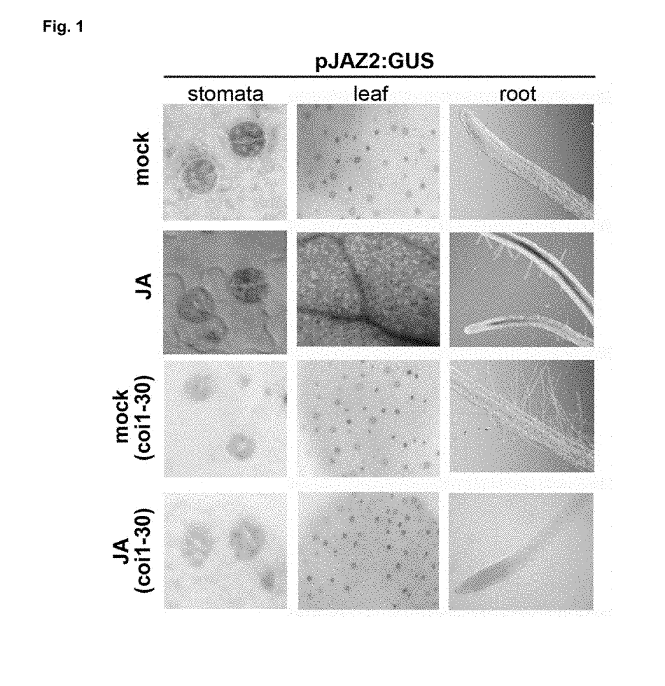

To identify JAZ genes specifically expressed at stomata, inventors produced transgenic Arabidopsis lines harboring a promoter construct covering around 2 Kb upstream of the ATG of each JAZ fused to the p-glucuronidase (GUS) reporter gene. It was successfully obtained stable transgenic plants expressing the corresponding JAZ promoter:GUS fusions for seven out of the twelve JAZ genes (JAZ1; AT1G19180, JAZ2; AT1G74950, JAZ3; AT3G17860, JAZ5; AT1G17380, JAZ6; AT1G72450, JAZ9; AT1G70700 and JAZ12; AT5G20900). Detailed examination of GUS expression driven by JAZ regulatory sequences at the stomata revealed that JAZ2 was the only one among these JAZs expressed in guard cells (FIG. 1). In basal conditions, expression of JAZ2 was exclusive of guard cells, but JA treatment induced it in roots and mesophyll cells of young seedlings, which partially masked the guard cell signal. Histochemical analyses of pJAZ2:GUS transgenics into the coi1-30 background (coi1-30-null mutant) revealed that basal expression of JAZ2 at the stomata occurs in a COI1-independent manner (FIG. 1). However, COI1 is required for JA-responsiveness of JAZ2 in mesophyll and root cells.

### Example 2

### JAZ2 Regulates Stomata Dynamics During Bacterial Pathogenesis

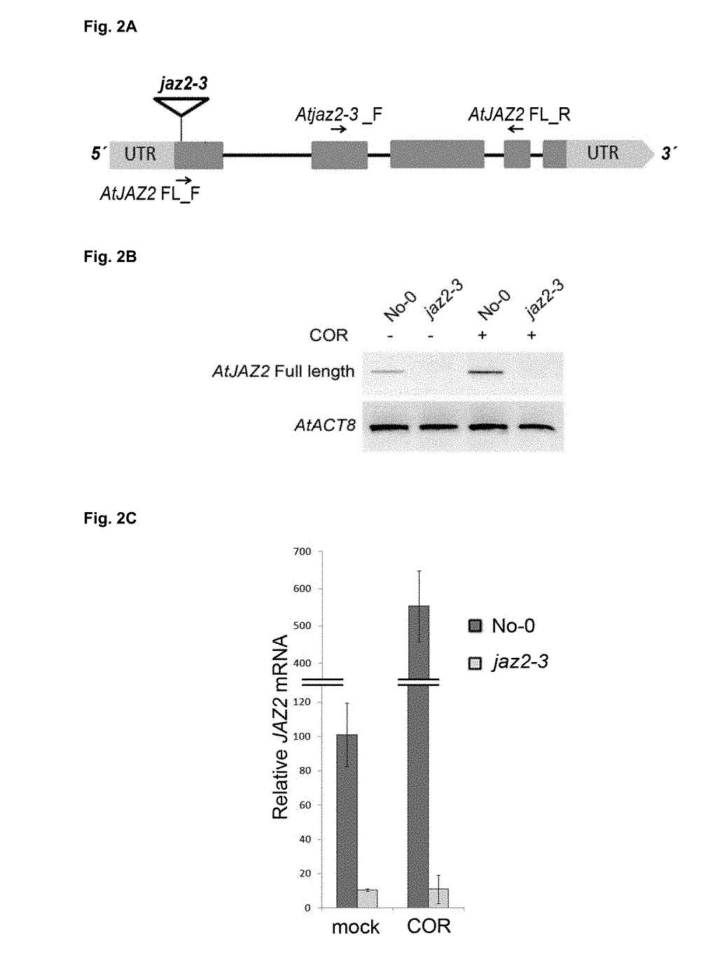

To test whether JAZ2 function as a regulator of stomatal dynamics during bacterial invasion, it was first obtained an Arabidopsis transposon insertion mutant, designated JAZ2-3 (SEQ ID NO: 12), in the accession Nossen (No-0), and selected homozygous plants (FIG. 2). Gene expression analyses supported that this mutant is a knock-out, or at least a knock-down, since it does not express the full-length JAZ2 gene, and expresses very low levels of truncated mRNA 3′ downstream the insertion (FIG. 2).

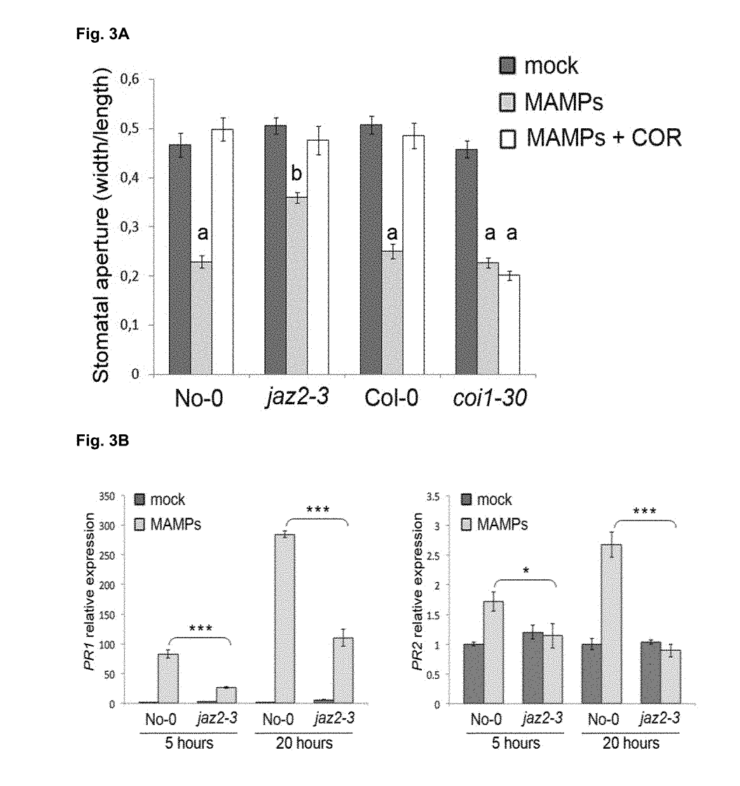

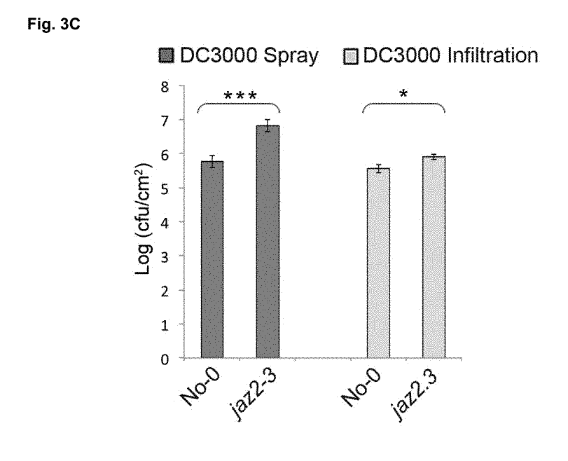

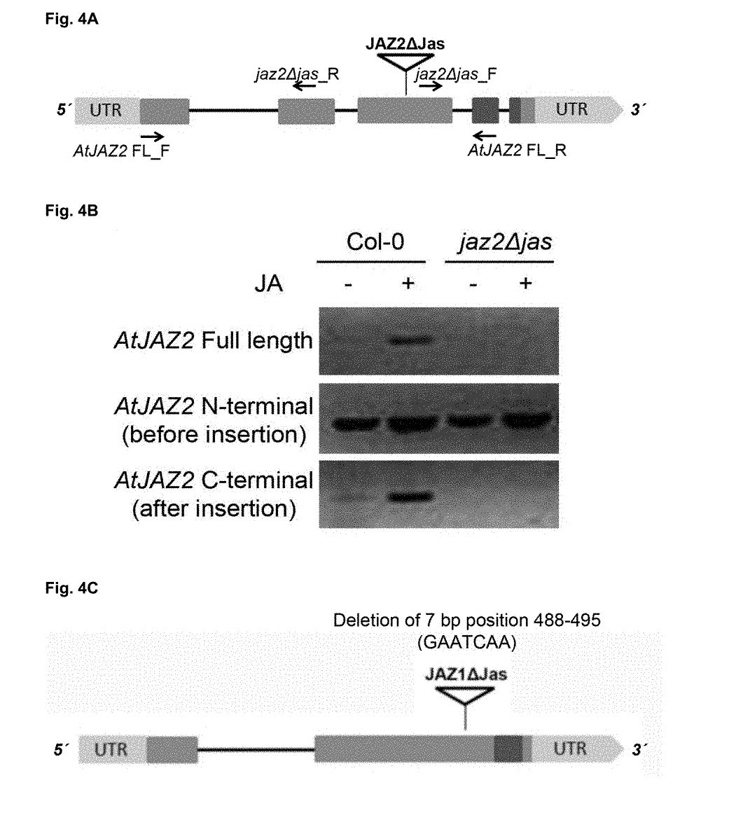

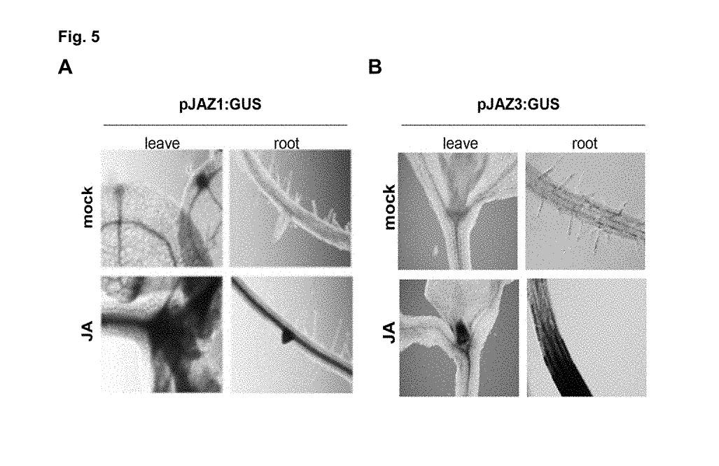

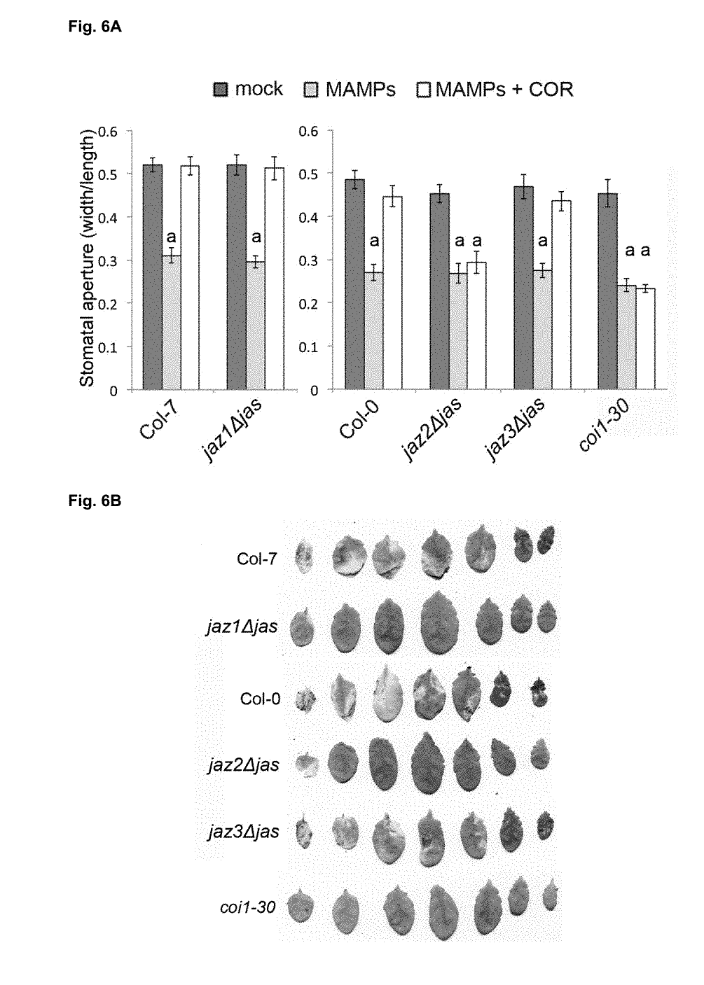

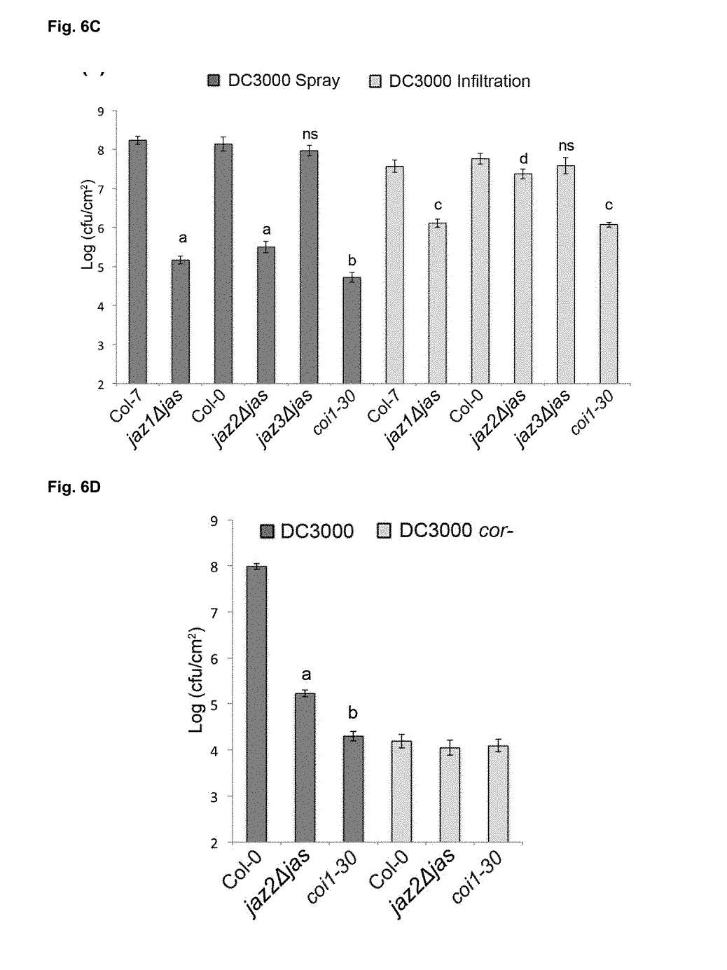

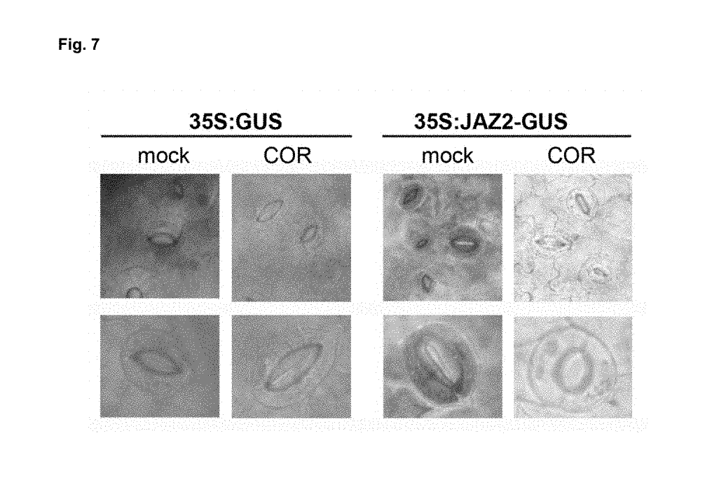

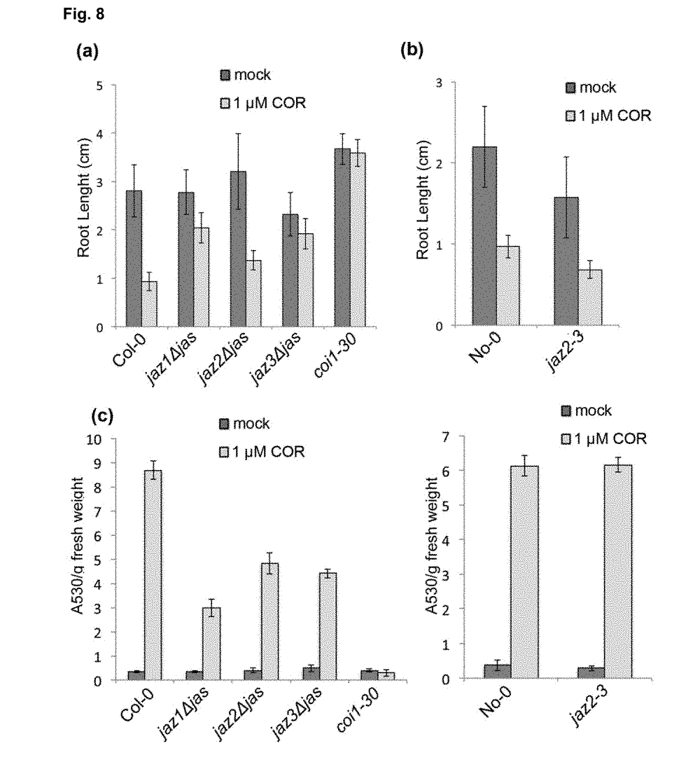

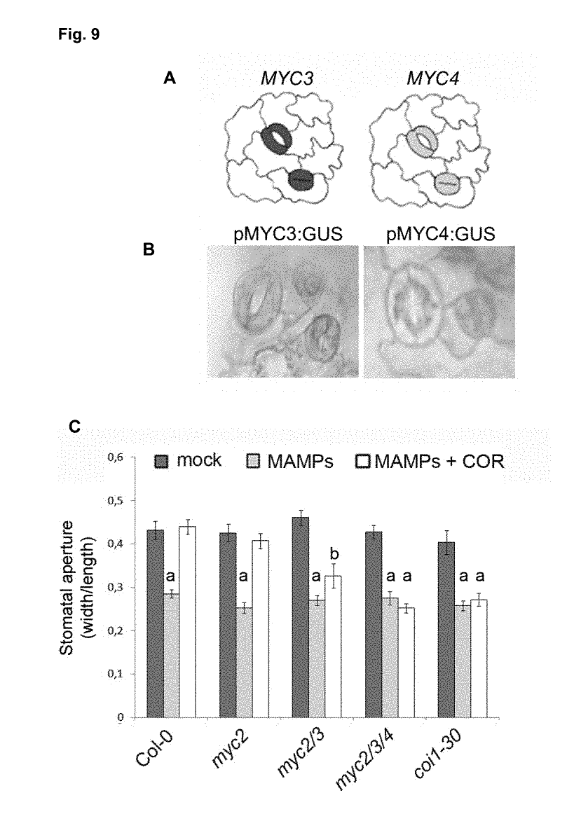

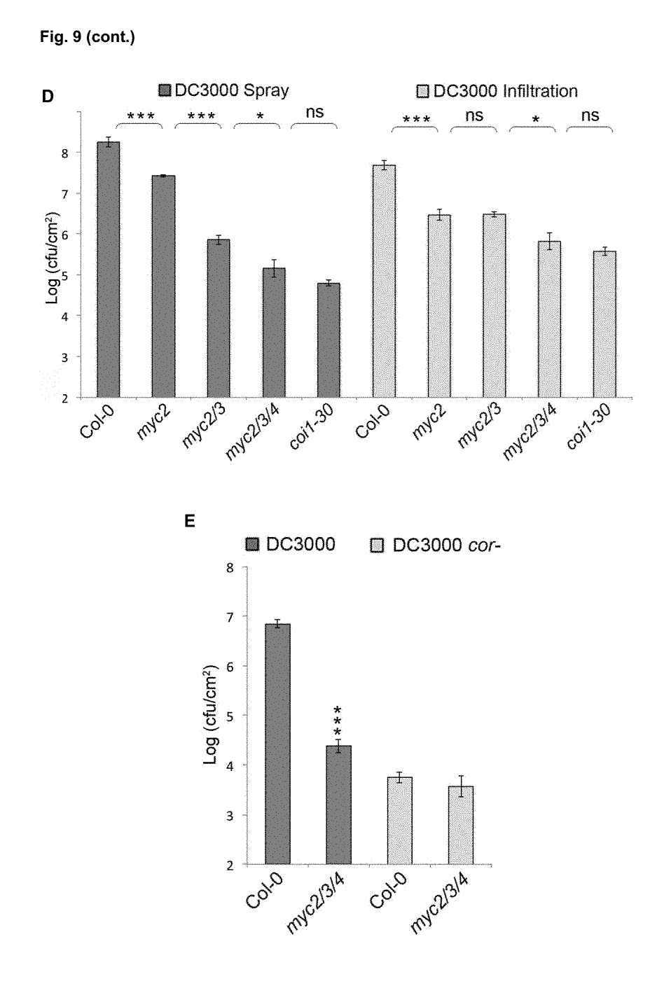

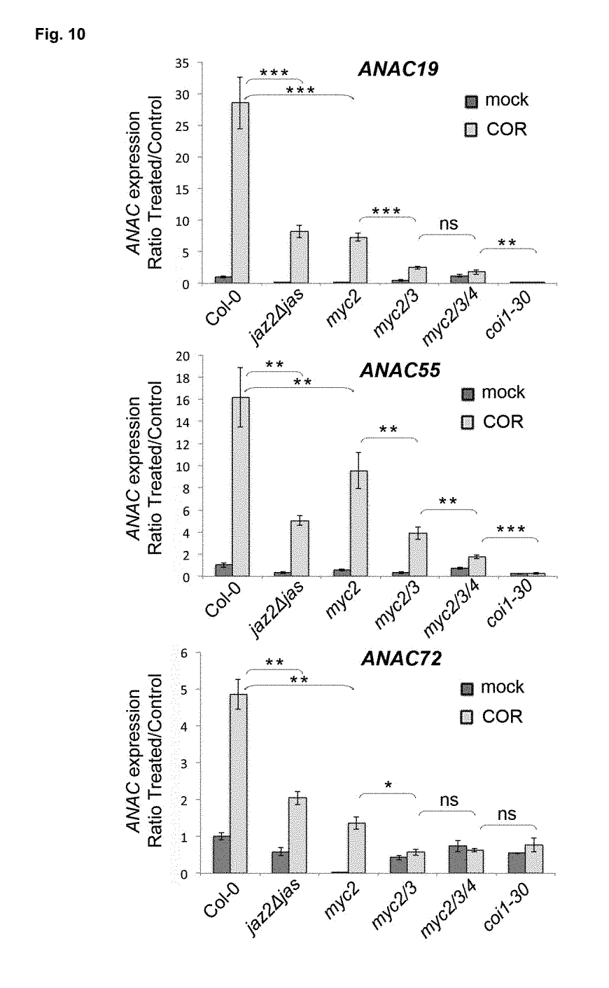

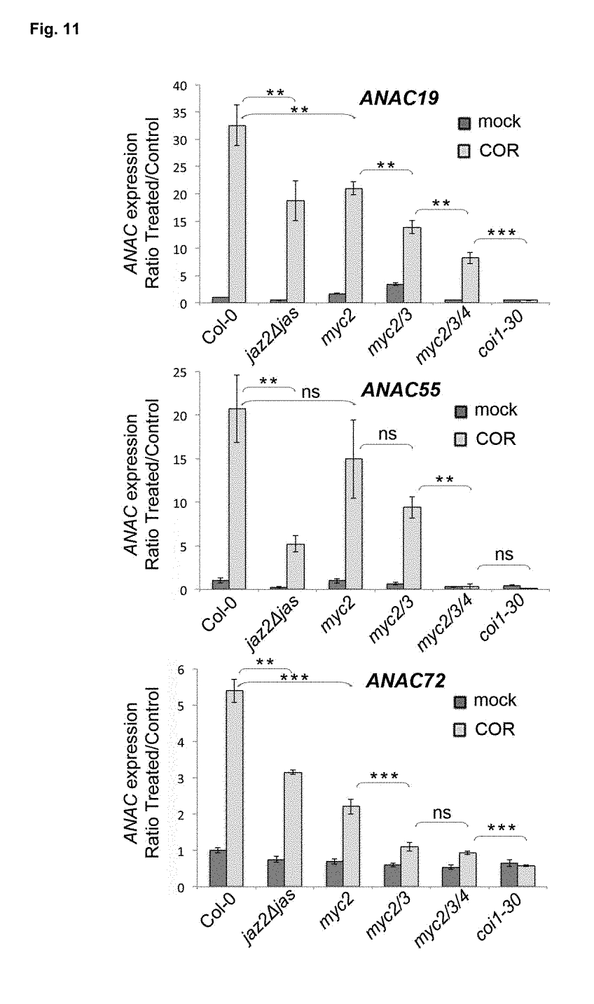

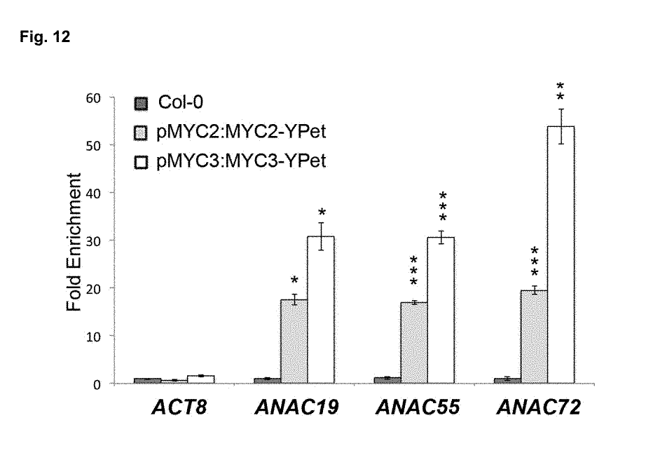

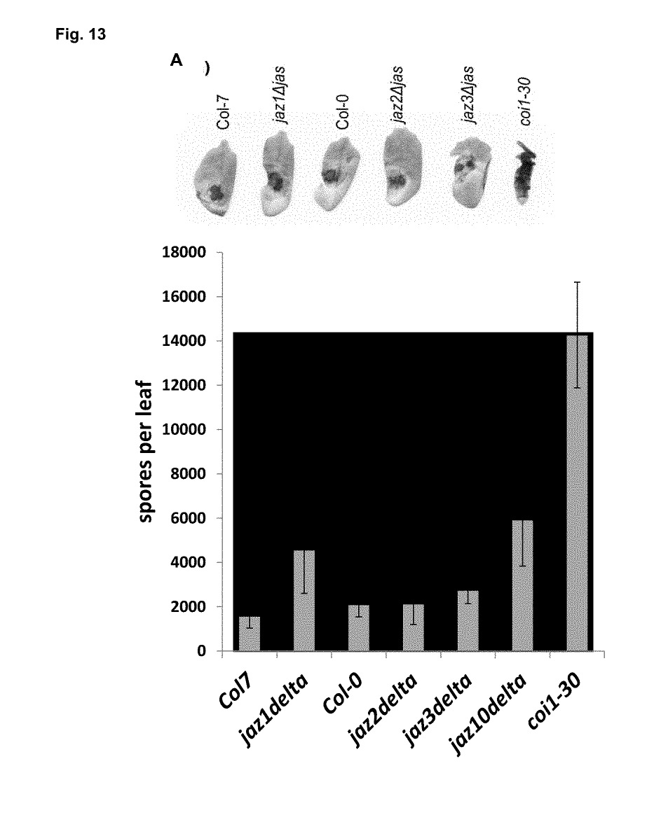

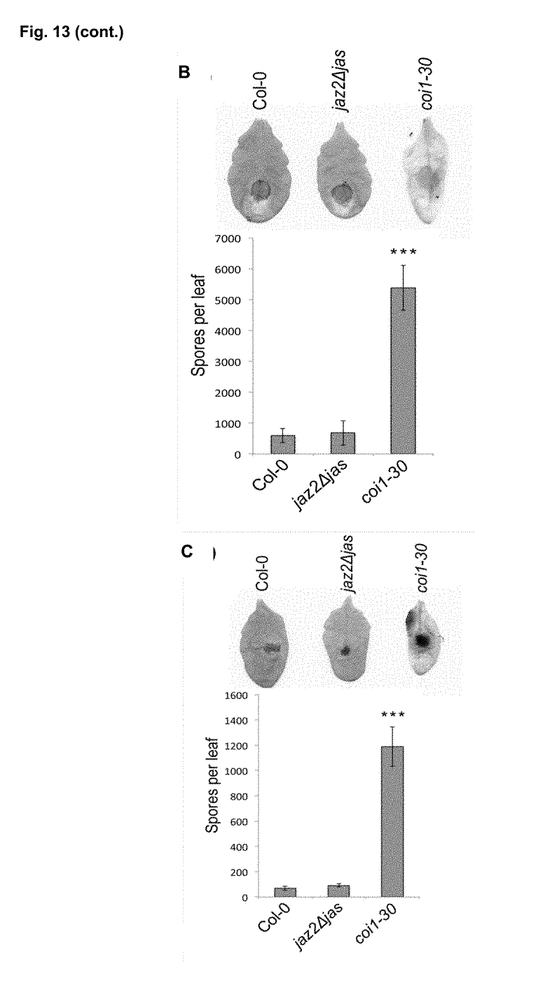

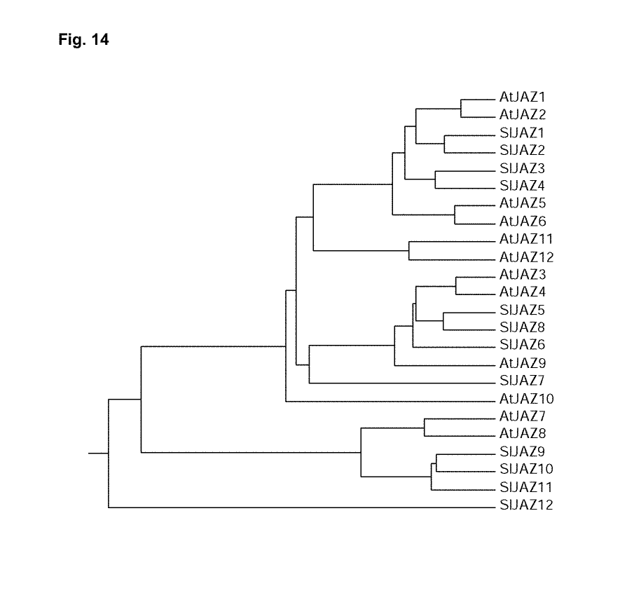

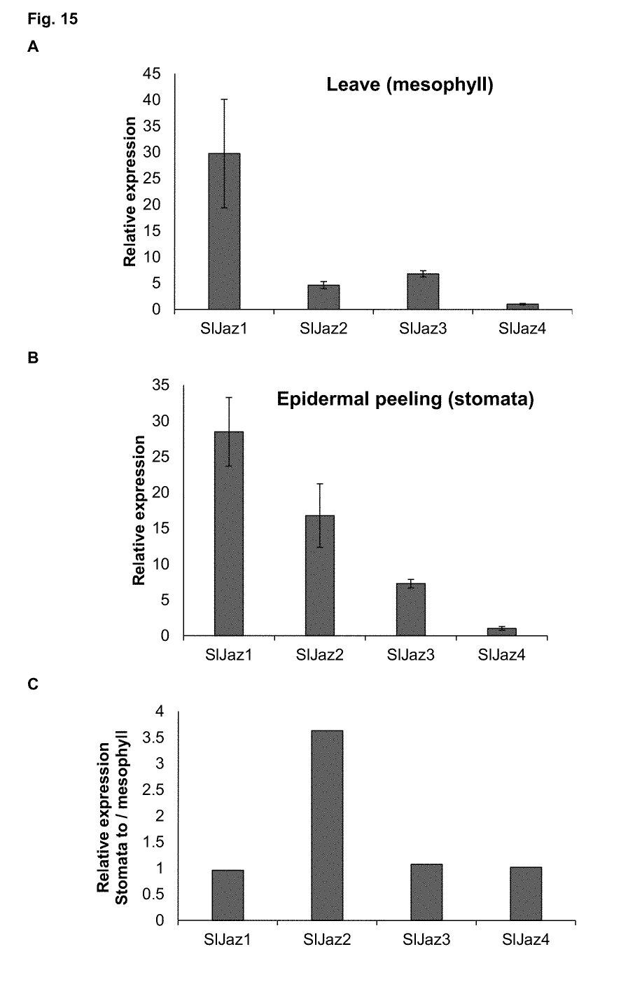

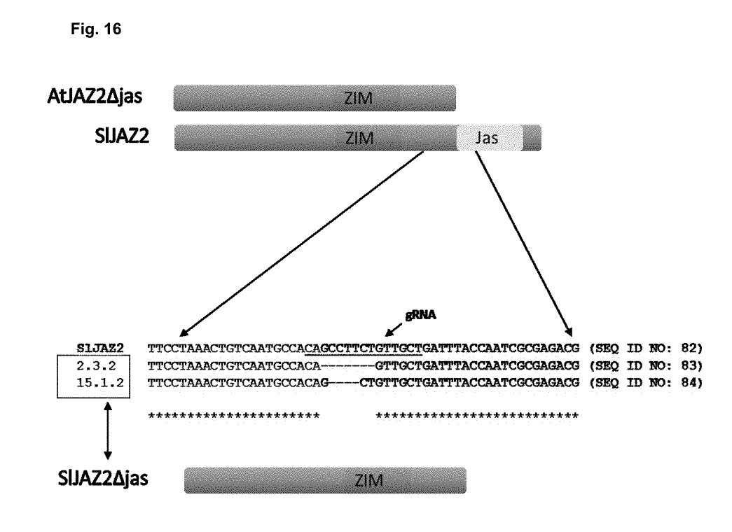

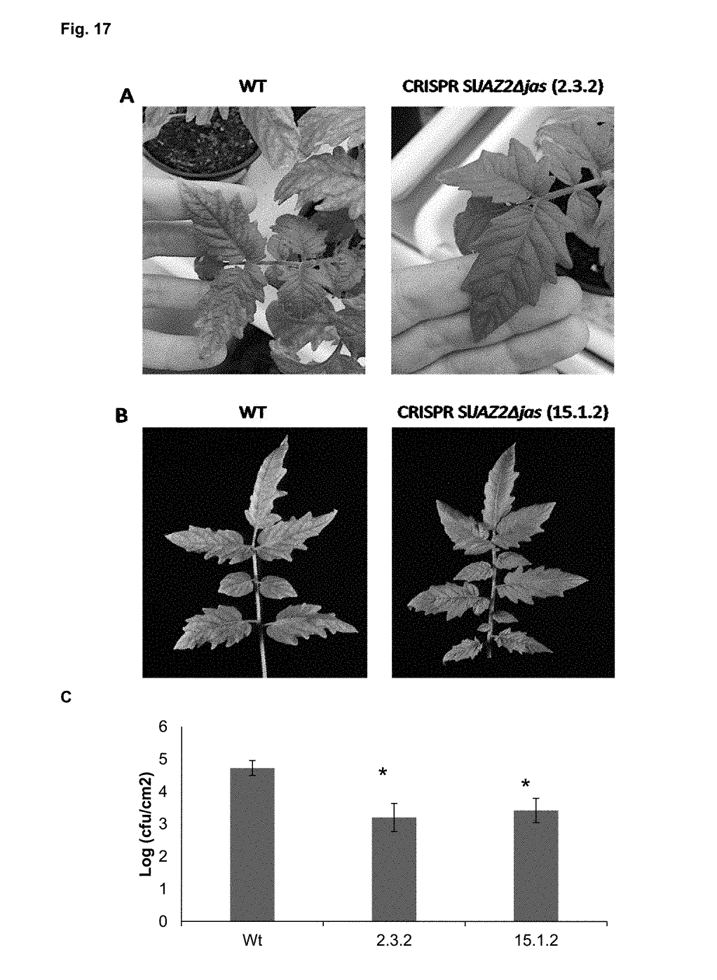

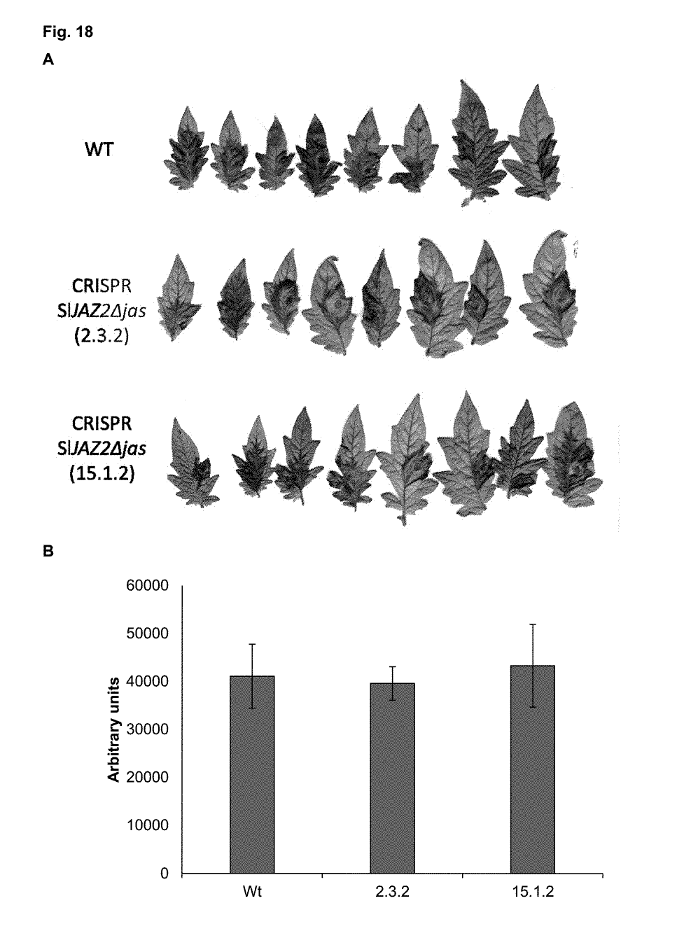

We next analyzed the ability of JAZ2-3 to close stomata upon microbial perception and reopen it in the presence of COR. MAMPs in crude boiled Pto DC3000 bacterial extracts induce stomata closing (Kunze, G. et al. Plant Cell. 2004; 16(12): 3496-3507; Gimenez-Ibanez, S. et al. Plant Signal Behay. 2009, 4(6): 539-54), whereas addition of COR to these extracts promote stomata reopening (Melotto, M. et al. Cell. 2006; 126(5): 969-980). Thus, whole leaves were incubated with bacterial extracts (MAMPs), MAMPs plus COR or a mock solution as a control. The crude Pto DC3000 MAMP extracts were prepared as previously described (Kunze, G. et al. Plant Cell. 2004; 16(12): 3496-3507; Gimenez-Ibanez, S. et al. Plant Signal Behav. 2009, 4(6): 539-54). Briefly, Pto DC3000 was grown in LB-medium at 28° C. on a rotary shaker until OD₆₀₀˜0.6-1.0. Bacteria were harvested by centrifugation, washed and resuspended in water (20 to 30% cells [fresh weight]/volume). Crude bacterial extracts were prepared by boiling the bacterial suspensions for 10 minutes and kept as cell containing Pto DC3000 MAMP crude extracts at −20 degrees. Typically, Pto DC3000 MAMPs were used at optical density at 600 nm OD₆₀₀=1,5 (7,5×108 cfu/ml) for incubation with leaves (Stomatal Aperture Measurements) or Arabidopsis seedlings (Quantitative RT-PCR). COR was purchased from Sigma-Aldrich and dissolved in EtOH. The results show that MAMPs induced stomatal closure in WT plants and coi1-30 mutants (coi1-30-null mutant). However, the single JAZ2-3 mutant comprising the transposon of SEQ ID NO: 12, obtained as it is mentioned above, was partially impaired in MAMP-induced stomata closing (FIG. 3A). Incubation of leaves with MAMPs and COR, simultaneously, triggered COR-induced stomata reopening in JAZ2-3 and WT plants, but not in coi1-30 (coi1-30-null mutant) (FIG. 3A). These results support previous observations indicating that COR-induced reopening of stomata is dependent on COR perception through the co-receptor COI1 (Melotto, M., et al. Cell. 2006; 126(5): 969-980), and suggests that JAZ2 plays a role in modulating stomata closure after bacterial perception.

Pathogen-induced stomatal closure in Arabidopsis depends on SA biosynthesis and signaling pathways (Melotto, M., et al. Cell. 2006; 126(5): 969-980; Zeng, W. and He, S. Y. Plant Physiol 2010; 153(3): 1188-1198; Zeng, W. et al. PLoS Pathog. 2011; 7(10): e1002291; Arnaud, D. and Hwang, I. Mol Plant 2015; 8(4): 566-581). Thus, it was evaluated the ability of WT and the single JAZ2-3 mutant to activate SA signaling upon MAMP perception by monitoring the expression of the SA marker genes PR1 and PR2. MAMPs induced significantly PR1 and PR2 expression in WT plants after 5 or 20 hours of incubation (FIG. 3B). In contrast, MAMP induced expression of these genes was strongly compromised in the single JAZ2-3 mutant compared to its control mock treatment. This suggests that the compromised ability of plants lacking JAZ2 to trigger MAMP induced stomatal closure might be the consequence of a defective activation of SA signaling pathway upon pathogen perception.

In addition to induce stomatal reopening, COR is also essential for the bacteria to overcome the mesophyll cell-based defenses occurring in the apoplast (Cui, J. et al. Proc Natl Acad Sci USA. 2005; 102(5): 1791-1796; Laurie-Berry, N. et al. Mol Plant Microbe Interact. 2006; 19(7): 789-800; Zeng, W., et al. PLoS Pathog. 2011; 7(10): e1002291; Zheng, X. Y. et al. Cell Host Microbe. 2012; 11(6): 587-596). To differentiate the contribution of JAZ2 to bacterial defense by either regulating stomata apertures and/or mesophyll cell-based defences in the apoplast, it was compared bacterial replication of P. syringae pv. tomato DC3000 (Pto DC3000) on WT (No-0) and JAZ2-3 Arabidopsis plants infected by spray inoculation or syringe infiltration (Zipfel, C. et al. Nature. 2004; 428(6984): 764-767). The spray inoculation technique mimics natural infection conditions and is one of the most sensitive techniques to assess plant susceptibility to bacterial pathogens (Zipfel, C. et al. Nature. 2004; 428(6984): 764-767). In contrast, the syringe infiltration bypasses the early stomatal level of regulation measuring mainly apoplastic cell-based defences. Three days post-spray inoculation bacterial titers in JAZ2-3 plants were significantly higher than those in its respective WT control. However, the titers were similar in JAZ2-3 and WT after infiltration (FIG. 3C). These results indicate that JAZ2 negatively regulates disease resistance to Pseudomonas bacteria and support a major role for JAZ2 in early penetration stages of the bacteria through the stomata compared to the weak role of JAZ2 in apoplastic cell-based defenses.

### Example 3

### Dominant Negative JAZ2ΔJas Mutants are Impaired in COR-Induced Stomata Re-Opening and are Resistance to P.syringae Infections

To study the specific function of JAZ2 at stomata analyses of dominant-negative JAZ variants were also performed. Truncated JAZ forms lacking the C-terminal Jas domain (JAZΔJas) are resistant to COI1-dependent degradation and behave as constitutive active repressors, blocking the activity of TFs and conferring JA-insensitivity (Chini, A. et al. Nature. 2007; 448(7154): 666-671; Katsir, L. et al. Proc Natl Acad Sci USA. 2008; 105(19): 7100-7105; Sheard, L. B. et al. Nature. 2010; 468(7322): 400-405; Moreno, J. E. et al. Plant Physiol. 2013; 162(2): 1006-1017). To further evaluate the effect of these gain-of-function repressors in stomatal dynamics inventors searched for Arabidopsis T-DNA insertion mutants that would result in JAZ2ΔJas forms under the control of its natural genomic context. It was identified a T-DNA line (GABI collection) that contained an insertion in the third exon (FIGS. 4A and B), and therefore, translated JAZ2 into an aberrant protein lacking the C-terminal Jas domain, this mutant was designated as JAZ2ΔJas (SEQ ID NO: 10).

As a control for specificity, it was also included in these analyses other JAZΔJas mutants from JAZ genes not expressed at stomata. Thus, was used the previously described jai3-1 dominant mutant (hereafter named JAZ3ΔJas, SEQ ID NO: 14 for the cDNA and SEQ ID NO: 15 for the aminoacid sequence) (Chini, A. et al. Nature. 2007; 448(7154): 666-671). JAZ3 is prevalently expressed in roots, both in basal and JA induced conditions, but is not expressed at stomata (FIG. 5B). See SEQ ID NO: 13 for the promoter of JAZ3. Additionally, a newly identified JAZ1ΔJas (SEQ ID NO: 60 for the cDNA and SEQ ID NO: 61 for the aminoacid sequence) in the Arabidopsis accession Col-7 (FIG. 4C) also behaves as a dominant mutant. JAZ1 was also expressed in roots in basal conditions whereas JA treatment strongly induced JAZ1 in roots and mesophyll cells (FIG. 5A). Therefore, JAZ1 (FIG. 5A), JAZ2 (FIG. 1) and JAZ3 (FIG. 5B) have different expression patterns, which suggest that they may have diverse functional specificities.

To assess the effect of these different mutations in stomatal and apoplastic immunity, whole leaves of JAZ1ΔJas (SEQ ID NO: 61), JAZ2ΔJas (SEQ ID NO: 10) and JAZ3ΔJas (SEQ ID NO: 15) mutants, and their respective WT backgrounds were incubated with MAMPs, MAMPs plus COR or a mock control, as described above. Similar to WT and coi1-30 plants, the dominant-negative version of JAZ1 (JAZ1ΔJas), JAZ2 (JAZ2ΔJas) and JAZ3 (JAZ3ΔJas) closed stomata when incubated with MAMPs, indicating that these repressive truncated JAZs forms do not compromise stomata closing upon microbial perception (FIG. 6A). When leaves were incubated with MAMPs and COR simultaneously, WT, JAZ1ΔJas and JAZ3ΔJas Arabidopsis plants induced COR-mediated stomatal reopening as expected (FIG. 6A). However, similar to coi1-30, JAZ2ΔJas mutants were fully impaired in COR-mediated stomatal reopening. This indicates that JAZ2 regulates stomata closure and that COR-induced stomata reopening require inhibition of JAZ2.

It was next compared bacterial replication of Pto DC3000 on Arabidopsis WT (Col-0), JAZ1ΔJas, JAZ2ΔJas, JAZ3ΔJas and coi1-30 plants infected by spray inoculation or syringe infiltration. Infections with sprayed Pto DC3000 bacteria showed similar symptom development on WT and JAZ3ΔJas plants (FIG. 6B). In contrast, JAZ1ΔJas and JAZ2ΔJas were remarkably more resistant than WT and resembled coi1-30 mutants, with very few chlorotic symptoms typical of Pseudomonas infections (FIG. 6B). Plant symptomatology correlated well with bacterial titers. Similar to coi1-30 plants, JAZ1ΔJas and JAZ2ΔJas leaves sprayed with Pto DC3000 contained remarkably lower bacterial titers than WT, whereas bacterial counts in JAZ3ΔJas plants were significantly higher and close to WT levels (FIG. 6C). Still, bacterial levels in JAZ2ΔJas leaves were always slightly higher than coi1-30 plants. There were next performed Pto DC3000 infection assays by syringe infiltration of Pto DC3000 into the apoplast. JAZ3ΔJas leaves contained similar bacterial counts as those observed in WT (Col-0) plants when Pto DC3000 bacteria was sprayed onto the leave, indicating that JAZ3 does not play a major role in regulating COR-induced stomatal aperture nor apoplastic defense responses in aerial tissues (FIG. 6C). In contrast, JAZ1ΔJas leaves contained still lower bacterial counts compared to WT plants when Pto DC3000 bacteria were infiltrated onto the leaf, suggesting that JAZ1 plays a major role in the regulation of apoplastic defense responses (FIG. 6C). Remarkably, disease susceptibility could be restored in the JAZ2ΔJas mutant when bypassing stomata regulation through syringe injection of bacteria (FIG. 6C). Indeed, differences in bacterial growth observed when Pto DC3000 bacteria was sprayed were significantly diminished when the same bacteria was injected into the leave, supporting the idea that JAZ2 functions during the early penetration process of Pto DC3000 bacteria by specifically regulating stomatal aperture. Still, JAZ2ΔJas leaves typically contained slightly less bacterial counts than WT plants which reflect a minor effect of JAZ2 on aploplastic defense.

To further support the idea that COR contributes to promote bacterial pathogenicity by targeting JAZ2, there were sprayed Pto DC3000 or the COR-deficient Pto DC3000 cor-bacteria onto WT, JAZ2ΔJas and coi1-30 plants. As previously observed, three days after spray with Pto DC3000, WT leaves contained higher bacterial titers than JAZ2ΔJas and coi1-30 mutants (FIG. 6D). In contrast, all WT, JAZ2ΔJas and coi1-30 plants exhibited similar levels of bacterial growth when the COR-deficient Pto DC3000 cor- was sprayed onto the leaves (FIG. 6D). These results indicate again that the virulence effect of COR during the bacterial infective process requires elimination of JAZ2, and therefore, cannot be exerted in this constitutively active (stable) variant of JAZ2, which is resistant to degradation. Supporting this, further experiments to determine JAZ2 protein stability in the presence of COR at stomata indicated that concentrations as low as 50 nM of COR induced the rapid degradation of JAZ2 protein at guard cells in transgenic Arabidopsis plants ectopically expressing JAZ2 fused to the GUS reported gene (FIG. 7). Altogether, these data support a prominent role of JAZ2 in stomatal movement regulation during the infection process of phytopathogenic Pseudomonas bacteria.

### Example 4

### JAZ2 has a Minor Role in JA-Responses Outside Guard Cells

To further support the specificity of JAZ2 in guard cell regulation, it was analyzed other typical JA-regulated responses, such as root-growth inhibition and anthocyanin accumulation. As shown in FIG. 8, root-growth inhibition in response to COR was unaffected in either JAZ2-3 or JAZ2IJas plants. In contrast, JAZ1ΔJas and JAZ3ΔJas were markedly insensitive to COR, which is consistent with their expression patterns (FIG. 8A and B). Regarding anthocyanin accumulation, JAZ1ΔJas plants were severely compromised in their ability to accumulate anthocyanin in response to COR, whereas JAZ2ΔJas and JAZ3ΔJas dominant JAZ versions showed a milder decrease in anthocyanin accumulation compare to JAZ1ΔJas plants (FIG. 8C). JAZ2-3 mutant plants showed completely normal anthocyanin accumulation in response to COR (FIG. 8C).

### Example 5

### MYC2, MYC3 and MYC4 Regulate Redundantly COR-Mediated Stomatal Reopening

COR-induced stomatal reopening and apoplastic defense is achieved through direct activation of ANAC19, ANAC55 and ANAC72 by the TF MYC2 (Zheng, X. Y. et al. Cell Host Microbe. 2012; 11(6): 587-596). However, COR-induced NAC induction is not completely abolished in a myc2 mutant (Zheng, X. Y. et al. Cell Host Microbe. 2012; 11(6): 587-596), suggesting that additional TFs should play a redundant function with MYC2 in regulating COR-induced stomatal reopening. MYC3 and MYC4 are also targets of JAZ repressors, JAZ2 among others, and regulate redundantly with MYC2 some JA-Ile-dependent responses, including pathogen resistance (Cheng, Z. et al. Mol Plant. 2011; 4(2): 279-288; Fernandez-Calvo, P. et al. Plant Cell. 2011; 23(2): 701-715; Niu, Y. et al. J Exp Bot. 2011; 62(6): 2143-2154; Moreno, J. E. et al. Plant Physiol. 2013; 162(2): 1006-1017). To investigate whether MYC3 and/or MYC4 could play a redundant role with MYC2 in regulating COR-mediated stomata reopening, inventors first examined the tissue-specific expression patterns of these TFs. Gene expression data in public databases (like http://bbc.botany.utoronto.ca/efp) indicated that MYC3 and MYC4 are expressed in mature guard cells, although the expression of MYC4 was much lower (FIG. 9A). Promoter-GUS fusion assays of MYC3 and MYC4 confirmed these data (FIG. 9B).

It was next analyzed the ability of single myc2, double myc2myc3 and triple myc2myc3myc4 mutants to induce COR-mediated stomatal reopening. It was included in these experiments coi1-30 as a negative control. As expected, all mutants and WT plants closed stomata when incubated with MAMP extracts (FIG. 9C). However, in the presence of MAMPs and COR, myc2myc3myc4 mutants could not reopen stomata in a similar fashion as coi1-30 (FIG. 9C). myc2myc3 mutants showed an intermediate phenotype, whereas the single myc2 mutant induced COR-mediatd stomatal reopening similar to WT plants. Altogether, these results indicate that depletion of these three MYCs is sufficient to render plants fully insensitive to COR in terms of stomata reopening and that MYC2, MYC3 and MYC4 play redundant roles in controlling COR-induced stomata reopening.

COI1 regulates both apoplastic defences and stomatal re-opening (Melotto, M., et al. Cell. 2006; 126(5): 969-980; Zheng, X. Y. et al. Cell Host Microbe. 2012; 11(6): 587-596). Bacterial growth after infiltration or spray inoculation was very similar in myc2myc3myc4 and coi1-30, further supporting that these MYCs also regulate both processes (FIG. 9D). Consistent with a redundant function, the single myc2 and double myc2myc3 mutants showed an intermediate phenotype between WT and the triple myc2myc3myc4 when bacteria were spray inoculated onte the leaves or directly infiltrated into the apoplast (FIG. 9D). These results are consistent with the effects on myc2, myc3 and myc4 mutants on stomatal aperture. Moreover, it was also analyzed the extent to which COR contributes to promotion of bacterial pathogenicity through these MYCs. When COR-deficient Pto DC3000 COR-bacteria was sprayed onto WT and myc2myc3myc4 plants, both exhibited rather similar levels of bacterial growth compared to the almost three log (cfu/cm²) difference that it was typically observed when Pto DC3000 was sprayed onto the same plants (FIG. 9E), supporting the notion that the virulence effect of COR is mediated by these three MYCs.

Altogether, these results suggest that MYC2, MYC3 and MYC4 act redundantly controlling both COR-induced stomata reopening and apoplastic defenses.

### Example 6

### MYC2, MYC3, MYC4 and JAZ2 Control COR-Dependent Expression of ANAC19, ANAC55 and ANAC72

Next it was evaluated whether MYCs regulate ANAC gene expression. Inventors monitored expression of ANAC19, ANAC55 and ANAC72 in myc mutant seedlings, such as, JAZ2ΔJas, myc2, myc2myc3 and myc2myc3myc4 mutants treated for 5 and 20 hours with COR or a mock solution. As shown in FIGS. 10 and 11, COR-induced expression of ANAC19, ANAC55 and ANAC72 was severely compromised in the JAZ2ΔJas, myc2 and myc2myc3 mutants and almost completely abolished in the triple myc2myc3mcy4 mutant. These results indicate that MYC3 and MYC4 are required for full COR-dependent induction of ANAC19, ANAC55 and ANAC72 and act additively with MYC2. Moreover, the results also support that JAZ2 is a repressor of ANAC gene expression mediated by MYCs in response to COR. These results are consistent with the phenotypic data showing increased bacterial susceptibility and impaired stomatal aperture in the myc mutants (FIG. 9). Moreover, they suggest that these three MYCs are sufficient to explain almost all COI1-dependent regulation of COR-induced stomatal reopening and apoplastic defences.

MYC2 can bind the promoter of ANAC genes and regulate their expression when overexpressed in transgenic plants. Since overexpression might be prone to artifacts, the inventors tested whether MYCs regulated by their native promoters could bind directly to the promoter of ANAC genes. Thus, we performed chromatin immuno-precipitation (ChlP)-PCR experiments using transgenic Arabidopsis plants expressing a MYC2-YPet-3xHA or MYC3-YPet-6xHis-3xFLAG fusion protein from their native genomic context. These were generated using recombineering methodology to insert seamlessly tags immediately before each gene stop codons. The results of ChlP-PCR showed that MYC2 and MYC3 bind efficiently to the promoters of ANAC19, ANAC55 and ANAC72 genes but not to control gene ACT8 (FIG. 12). These results indicate that both MYC2 and MYC3 activate the expression of the three ANACs by direct interaction with their promoters under their natural genomic contexts.

Altogether, our data indicate that COR-COI1-JAZ2-MYC2/3/4-NAC19/55/72 forms a signaling module controlling stomatal responses during the invasive process of phytopathogenic Pseudomonas syringae, whereas other JAZs would likely form such modules to regulate apoplastic defenses.

### Example 7

### The JAZZ2ΔJas Mutant Shows Unaltered Levels of Resistance Against the Necrotrophic Pathogens

JA and SA defense pathways generally antagonize each other and thus, strategies to enhance apoplastic defenses against necrotrophs often lead to increased susceptibility to biotrophs, and vice versa (Grant, M. and Lamb, C. Curr Opin Plant Biol. 2006; 9(4):414-20). The cell specific JAZ2 function at guard cells implies that JAZ2ΔJas mutation should not compromise apoplastic defences to necrotrophs which invade the plant tissue by active penetration through enzymes and/or appressoria-like structures that establish a primary lesion through which the pathogen can penetrate into the host surface. To test this, it was measured susceptibility of WT, JAZ1ΔJas, JAZ2ΔJas, JAZ3ΔJas and coi1-30 plants to the necrotrophic fungi B. cinerea. Since JAZ1 is expressed in leaves and roots (FIG. 5), JAZ3 is preferentially expressed in roots (FIGS. 5) and COI1 is widely expressed in most tissues, therefore JAZ1ΔJas, JAZ3ΔJas and coi1-30 mutants are appropriate controls. Consistent with published data, six days after B. cinerea infection coi1-30 Arabidopsis plants showed enhanced fungal symptoms and increased spore production compared to control plants (FIG. 13). Consistent with JAZ1 expression in leaves, JAZ1ΔJas and JAZ10ΔJas (Moreno, JE. et al. Plant Phys. 2013; 162: 1006-1017. Line 14.4 pJAZ10:HA-JAZ10.4) also supported significantly higher number of spores compared to WT plants despite it was not comparable to the levels of susceptibility of coi1-30 plants. In contrast, JAZ2ΔJas and JAZ3ΔJas behave similarly to WT in terms of fungal symptoms and spore production (FIG. 13A), indicating that the JAZ2ΔJas mutation does not alter apoplastic susceptibility to the necrotrophic fungus B. cinerea, but not other JAZΔJas forms such as JAZ1ΔJas and JAZ10ΔJas. Indeed, it was often observed that the number of spores in JAZ2ΔJas leaves were even lower than in WT, suggesting that B. cinerea may also use stomata to favorfungal invasion.

Analyses of other necrotrophic fungi such as Plectosphaerella cucumerina (FIG. 13B) and Alternaria brassicicola (FIG. 13C) gave similar results, indicating that JAZ2ΔJas retains WT levels of resistance to a broad range of necrotrophs. Six days after P. cucumerina and A. brassicicola infection coi1-30 plants showed severe fungal symptoms and increased spore production compared to WT plants. In contrast, JAZ2ΔJas resembled WT plants with similar symptoms and content of spores in leaves (FIG. 13 B,C).

These results further support a key role of JAZ2 in regulating stomatal dynamics, but only a minor role in apoplastic defence responses. Moreover, these results suggest novel strategies for crop protection by manipulating JA/COR-dependent signaling events at the entry ports of specific microbes through the expression of JAZΔJas forms at guard cells. These new strategies will increase resistance to biotrophs without affecting susceptibility to other general pathogens due to the well-known antagonism between JA and SA.

By the improved understanding of the SA and JA defense pathways that has been achieved by the inventors, the present invention enhances the development of methods for improving the tolerance of plants to biotrophic pathogens (i.e. P. syringae) without enhancing susceptibility to necrotrophs (i.e. B. cinerea, P. cucumerina, A. brassiccicola), as well as for developing easier and more efficient methods for identifying pathogen-tolerant plants. The plants that can be developed using the methods of the present invention have a broad-spectrum resistance to biothrophs but without compromising their resistance to necrotrophs, and provide more and better plant production and consequently plant products for market in both developed and underdeveloped countries.

As conclusion, the results showed in the present invention show that phytopathogenic Pseudomonas produces COR to hijack a COI1-JAZ2-MYC2/3/4-NAC19/55/72 signaling module controlling stomatal responses during the invasive process. Cell-specific expression of JAZ2 at guard cells state that JAZ2 regulates stomatal dynamics during bacterial invasion. Loss- and gain-of-function analyses using JAZ2-3 and JAZ2ΔJas mutants after infiltration or spray inoculation confirmed these results, and supported the fact that JAZ2 primarily functions at the guard cells. The gain-of-function JAZ2ΔJas allele blocked stomatal reopening and increased resistance to Pseudomonas, whereas the loss-of-function JAZ2-3 was partially impaired in stomatal closure and more susceptible to the bacteria. This partial phenotype of JAZ2-3 in stomatal closure could be due to redundancy among JAZ proteins. In fact, COR can still be perceived in the JAZ2-3 mutant at the stomatal guard cells suggesting that in these cells other JAZ proteins should form co-receptor complexes with COI1, in addition to JAZ2. The inventors succeed in analyzing the expression patterns of seven JAZs only, and therefore, a role for the remaining JAZs (JAZ4, JAZ7, JAZ8, JAZ10, JAZ11 and JAZ13) at the stomata could be expected.

Additionally, is also shown that JAZ2 plays a major role in controlling stomatal reopening during bacterial invasion and a minor (or redundant) role in apoplastic defenses. Firstly, the dominant JAZ2ΔJas plants were remarkably more resistant than WT when Pto DC3000 was sprayed onto the leaves, but only slightly more resistant when the stomata barrier was bypassed by leaf infiltration of the bacteria. Secondly, JAZ2ΔJas plants did not show altered resistance to necrotrophic fungi, which further supports that its repressive effect on JA signaling does not affect apoplastic defenses. Finally, further analysis of typical JA-mediated responses such as root-growth and anthocyanin accumulation are consistent with a prevalent role of JAZ2 in stomata since only a minor effect in anthocyanin accumulation was observed in dominant JAZ2ΔJas and none in JAZ2-3 loss-of-function mutants.

### Example 8

### Transference of JAZ2-Mediated Defence Resistance to Tomato (Solanum Licopersicum)

In order to show whether JAZ2-mediated resistance to P. syringae as demonstrated in A. thaliana can also be applicable to crop species, a commercial variety of tomato (Solanum licopersicum var. MoneyMaker) was elected to generate the equivalent mutation to Atjaz2Δjas by using genome editing technology.

Firstly the AtJAZ2 orthologue was identified by searching for JAZ genes in the tomato genome using BLAST. JAZ genes from Arabidopsis thaliana and Solanum lycopersicum were aligned with Dialing software (http://www.genomatix.de/cgi-bin/dialign/dialign.pl). Phylogenetic tree was represented using Phylodendron (http://iubio.bio.indiana.edu/treeapp/treeprint-form.html). Sequence alignment and phylogenetic analysis of the twelve identified tomato JAZ proteins showed that SIJAZ1 (SEQ ID NO: 78) and SIJAZ2 (SEQ ID NO: 79) were the tomato proteins more closely related to AtJAZ2, followed by SIJAZ3 (SEQ ID NO: 80) and SIJAZ4 (SEQ ID NO: 81) (FIG. 14). To further analyze whether any of these genes was expressed at stomata guard cells as AtJAZ2, gene expression patterns of these four tomato genes (SIJAZ1-4) we studied by using real time-PCR gene expression experiments in mesophyll cells (whole leaves) or epidermal peelings, which are enriched in stomata guard cells compared to whole leaves. The gene and protein sequences for sequence alignment and phylogenetic analysis have been found in the Database: Sol Genomics Network (SGN, www.solgenomics.net) and Plants Ensembel (www.plants.ensembl.org).

RNA was extracted using Favor prep Plant Total RNA Mini Kit (Favorgene, Taiwan). qRT-PCR was performed as described previously (Fernandez-Calvo P, et al. Plant Cell, 2011, 23: 701-7153). Table 1 shows the primers used in the present example.

TABLE 1
qRT-PCR primers

| qRT-PCR primers | Sequence (SEQ ID NO:) |
|---|---|
| SIActin FW | CAAGTTATTACCATTGGTGCTGAGA (SEQ ID NO: 62) |
| SIActin RV | TGCAGCTTCCATACCAATCATG (SEQ ID NO: 63 |
| SIJaz1 FW | GGAAACAATCCTGCTAAACCA (SEQ ID NO: 64) |
| SIJaz1 RV | TCCGAAACTCGGAACCAC (SEQ ID NO: 65) |
| SIJaz2 FW | AAGACAGAATCTTGGAAACCTGA (SEQ ID NO: 66) |
| SIJaz2 RV | AACAATGACTTGTCCACCATAAAA (SEQ ID NO: 67) |
| SIJaz3 FW | AACACCTCCAGATTAAGCCAGAC (SEQ ID NO: 68) |
| SIJaz3 RV | AATTGTGCTTGTGCTGTTGC (SEQ ID NO: 69) |
| SIJaz4 FW | TGGAAAAGCAAATATCAATGATCTAA (SEQ ID NO: 70) |
| SIJAZ4 RV | ACAAATCCTTTGTTGCTGAGG (SEQ ID NO: 71) |

As shown in FIG. 15A-B, SIJAZ1 was highly expressed in both stomata and guard cells, whereas SIJAZ3 and SIJAZ4 had a much lower expression in both tissues. In relative terms, SIJAZ1, SIJAZ3 and SIJAZ4 had a similar expression level in mesophyll and stomata guard cells, with expression ratios close to 1 (FIG. 15C). In contrast, SIJAZ2 had almost 4 fold higher expression in stomata guard cells compared to mesophyll cells, indicating that indeed SIJAZ2 is specifically enriched in guard cells as AtJAZ2. These results pinpointed SIJAZ2 as the functional orthologue of AtJAZ2.

Obtention of SIJAZ2ΔJas mutants using CRISPR/Cas9.

Genome editing has emerged as a technology with a potential to revolutionize plant breeding by introducing precisely favorable alleles into crops. To obtain a mutant in SIJAZ2 that lacks the Jas domain similar to the bacterial resistant AtJAZ2ΔJas form, CRISPR/Cas technology was used and a guide RNA (gRNA) oligonucleotide was designed at the beginning of the JAS sequence to guide the Cas9 cleavage of the gene (FIG. 16). Table 2 shows the primers used in the present example.

TABLE 2
Primers used as guide RNA.

| Primers used as guide RNA | Sequence (SEQ ID NO:) |
|---|---|
| 1° gRNA for SIJAZ2 (A) | ATTGAAATCAGCAACAGAAGGCTG |
| | (SEQ ID NO: 72) |
| 1° gRNA for SIJAZ2 (B) | AAACCAGCCTTCTGTTGCTGATTT |
| | (SEQ ID NO: 73) |
| 2° gRNA for SIJAZ2 (A) | ATTGCTGATTTACCAATCGCGAGA |
| | (SEQ ID NO: 74) |
| 2° gRNA for SIJAZ2 (B) | AAACTCTCGCGATTGGTAAATCAG |
| | (SEQ ID NO: 75) |

The corresponding constructs and transgenic tomato plants were generated by callus transformation and the mutants generated after regeneration by PCR were analysed, later the corresponding SIJAZ2 region was sequenced (SEQ ID NO: 82) (FIG. 16).

Plasmid construction was performed by the transformation of the plasmid pK7m34GW which can be retrieved from https://www.arabidopsis.org/servlets/TairObject?type=vector&id=1001200293, and later the gene that expresses Cas9 (regulated by the promoter of Ubiquitin 10) and gRNA (regulated by the promoter of Ubiquitin 6) was included in this plasmid.

Cloning was performed by ordinary methods. The tomato cultivar Moneymaker was transformed with the pK7_CAS9-TPC Sljaz2 as previously described (Wittmann, J., et al. Plant Pathol. 2016, 65: 496-502).

Extraction of genomic DNA was performed by extraction with NaCI, EDTA and Tris pH 8.0 and subsequent precipitation of DNA with isopropanol. Genomic DNA was amplified with primers for the Jas domain of SIJAZ2 and PCR products were sequenced on an ABI3730XL DNA sequencer. Table 3 shows the primers used in the present example.

TABLE 3

| Primers for | |
|---|---|
| sequencing SIJAZ2 | Sequence (SEQ ID NO:) |
| SIJaz2 Fw | ATCATGAAGTTAGCCAACAAACAG |
| | (SEQ ID NO: 76) |
| SIJaz2 Rv | GAAATATTGCTCAGTTTTAACAAATT |
| | (SEQ ID NO: 77) |

Two independent mutants were selected, namely 2.3.2 and 15.1.2 lines, that contained “out-of-frame” deletions at the beginning of the Jas motif and, therefore, should generate truncated proteins. Once identified CRISPR/Cas SIJAZ2ΔJas plants, homozygous plants for the two lines previously selected were obtained.

Resistance to bacterial and necrotrophic fungal pathogens of CRISPR/Cas SIJAZ2ΔJas plants.

As described above, Arabidopsis JAZ2AJas mutants are resistant to Pseudomonas syringae but retain unaltered resistance against necrotrophs, such as Botrytis cinerea.

Thus, the resistant of generated CRISPR/Cas SIJAZ2ΔJas plants to the hemibiotrophic tomato pathogen P. syringae pv. tomato DC3000 (Pto DC3000) or the necrotrophic fungi Botrytis cinerea, was analyzed. Four to six weeks-old plants were used for the infection assays. Those plants were grown in a cycle of 14 hours light and 10 hours darkness.

Pseudomonas syringae DC3000 infection assays

A suspension of DC3000 with an OD of 0.2 was used for plant infection. Plants were sprayed or dipped into the DC3000 solution. Leaf disks were collected seven days post infection and bacterial growth was quantified as described previously (Gimenez-Ibanez S, et al. Current Biology. 2009, 19: 423-429).

Seven days after spray-inoculation with Pto DC3000, WT tomato plants showed typically disease symptoms of Pseudomonas infections, characterized by specks surrounded by a chlorotic halo (FIG. 17A-B). In contrast, CRISPR/Cas SIJAZ2ΔJas plants hardly showed any visible disease symptoms (FIG. 17A-B). Consistently, bacteria titers in both CRISPR/Cas SIJAZ2ΔJas lines (2.3.2 and 15.1.2) were significantly lower compared to WT tomato plants. Indeed, WT plants sustain at least 10 fold more bacteria than both independent CRISPR/Cas SIJAZ2ΔJas lines (FIG. 17C) indicating that similar to Arabidopsis, elimination of the JAS domain of JAZ2 promotes bacterial resistance in tomato.

Botrytis Cynerea Infection Assays

Botritis cynerea infections were performed essentially as described previously (Monte I, et al. Nature Chemical Biology. 2014, 10: 671-676) with some modifications: 5 to 6 weeks tomato leaves were inoculated with 20 μl of a suspension of 5×10⁵ Botrytis spores ml-1 PDB (Difco, Le Pont de Claix, France). At least 8 leaves were inoculated per treatment. Disease symptoms were scored 4 days post inoculations. Area of the lesion was measured using jimage software (https://imagej.nih.gov/ij/).

In contrast to Pseudomonas syringae DC3000 infection, the infection with B. cinerea produced similar symptoms in WT and CRISPR/Cas SIJAZ2ΔJas plants, therefore indicating that this particular modification does not affect resistance to necrotrophic fungi (FIG. 18 A-B). The results demonstrate that genome editing technology can be successfully used to transfer basic knowledge generated in model plants to importantly economic crops towards more resistant crops against bacterial pests.

1. An isolated nucleic acid sequence that comprises a modified JAZ nucleic acid, operably linked to a promoter that regulates the specific expression of the nucleic acid in the guard cells of the stomata, wherein said modified JAZ encodes a polypeptide comprising a ZIM domain, but not a functional Jas motif; a functional variant of the modified JAZ/JAZ, a homologue or orthologue thereof.

2. The isolated nucleic acid according to claim 1 wherein the ZIM domain comprises SEQ ID NO: 27, preferably the ZIM domain comprises SEQ ID NO: 28.

3. The isolated nucleic acid according to claim 1 wherein the ZIM domain comprises the sequence SEQ ID NO: 4, preferably the ZIM domain comprises a sequence with at least 65% identity with SEQ ID NO: 5; a functional variant of the modified JAZ/JAZ, a homologue or orthologue thereof.

4. The isolated nucleic acid according to claim 1 wherein the Jas motif comprises the sequence SEQ ID NO: 6, preferably comprises a sequence with at least 80% identity with SEQ ID NO: 7; a functional variant of the modified JAZ/JAZ, a homologue or orthologue thereof.

5. The isolated nucleic acid according to claim 3 which comprises a sequence with at least 70% identity with SEQ ID NO: 8 or SEQ ID NO: 9; a functional variant of the modified JAZ/JAZ, a homologue or orthologue thereof.

6. The isolated nucleic acid according to claim 1 wherein the promoter comprising a nucleic acid sequence having at least 80% sequence identity to the promoter selected from the group consisting of promoter of JAZ2 and JAZ10, preferably the promoter comprising a nucleic acid sequence having at least 80% sequence identity to the promoter of JAZ2 of A. thaliana, more preferably the promoter is the SEQ ID NO: 11.

7. A host cell comprising the isolated nucleic acid according to claim 1, or the protein encoded by the isolated nucleic acid according to claim 1, wherein the cell is preferably a plant cell but not an Arabidopsis thaliana cell, more preferably the plant cell is a guard cell of the stomata.

8. A plant comprising the isolated nucleic acid according to claim 1, or the protein encoded by the isolated nucleic acid according to claim 1, or the host cell according to claim 7, wherein the plant is not an Arabidopsis thaliana plant.

9. A germplasm comprising the isolated nucleic acid according to claim 1, or the protein encoded by the isolated nucleic acid according to claim 1, or the host cell according to claim 7, wherein the germplasm is not an Arabidopsis thaliana germplasm.

10. A germplasm of the plant according to claim 8.

11. A use of the isolated nucleic acid according to claim 1, or the host cell according to claim 7, or the germplasm according to claim 9, to produce a plant with resistance to biotrophic or hemi-biotrophic plant pathogens, preferably Pseudomonas syringae, and without modifying the level of susceptibility of said plant to necrotrophic plant pathogens, preferably Botrytis cynerea.

12. A method for producing a plant with resistance to biotrophic or hemi-biotrophic plant pathogens, preferably to Pseudomonas syringae, and without modifying the level of susceptibility of said plant to necrotrophic plant pathogens, preferably Botrytis cinerea, comprising:
a. transferring to the isolated plant material the isolated nucleic acid sequence claim 1, wherein said transfer of said nucleic acid is performed by transformation, by gene gun, by electroporation, by viral transduction, by protoplast fusion, by a doubled haploid technique or by embryo rescue, provided that when said method involves a doubled haploid technique said method is not essentially biological,
b. identifying the plant material obtained in the step (a) comprising the modified JAZ nucleic acid operably linked to a promoter that regulates the specific expression of the nucleic acid in the guard cells of the stomata, as defined in claim 1,
c. growing the plant material identified in the step (b) in a suitable medium to produce at least a plant and/or a germplasm which expresses the modified JAZ nucleic acid sequence.

13. A method for producing a plant with resistance to biotrophic or hemi-biotrophic plant pathogens, preferably to Pseudomonas syringae, and without modifying the level of susceptibility of said plant to necrotrophic plant pathogens, preferably Botrytis cinerea, comprising:
a. modifying the JAZ native nucleic acid of the isolated plant material to obtain the nucleic acid encoding the non-functional Jas domain as defined in claim 1 by means of the Zinc finger nuclease 1 and 2 (ZFN1 and 2) technology, TALENs or CRISP/Cas technology;
b. identifying the plant material obtained in the step (a) having the nucleic acid encoding a non-functional Jas domain,
c. growing the plant material identified in the step (b) in a suitable medium to produce at least a plant and/or a germplasm which expresses the modified JAZ nucleic acid sequence.

14. A method for detecting a plant with resistance to biotrophic or hemi-biotrophic plant pathogens, preferably to Pseudomonas syringae, and without modifying the level of susceptibility of said plant to necrotrophic plant pathogens, preferably Botiytis cinerea, that comprises detecting the modified JAZ nucleic acid operably linked to a promoter that regulates the specific expression of the nucleic acid in the guard cells of the stomata, as defined in claim 1.


SEQUENCE LISTING (84 sequences)

SEQ ID NO: 1 - DNA, 750 bp, Arabidopsis thaliana
atgtcgagtttttctgccgagtgttgggacttctctggtcgtaaaccgagcttttcacaaacatgtactcgattgagtcgttacctgaaggagaagggtagttttggagatctgagcttagggatgacatgcaagcccgacgttaatggaggttcacgtcagcctacaatgatgaatctgttcccttgtgaagcttcaggaatggattcttctgctggtcaagaagacattaaaccgaagactatgtttccgagacaatcaagcttttcttcttcctcttcctctgggaccaaagaagatgtacagatgatcaaagagactactaaatctgtgaagccagagtctcaatctgctccgttgactatattctacggtggtcgagttatggtgtttgatgatttttctgctgagaaagctaaagaagtcattgatttggctaacaaaggaagtgccaaaagcttcacatgtttcacagctgaagtaaacaataaccatagtgcttattctcaaaaagagattgcttctagcccaaatcctgtttgtagtcctgcaaaaaccgcagcacaagagccaattcagcctaacccggcctctttagcctgcgaactcccgattgcaagaagagcttcacttcatcggttccttgagaagaggaaggataggatcacatcaaaggcaccataccaaatagacggttcagctgaagcgtcttccaagcctactaacccagcttggctcagttcacggtaa

SEQ ID NO: 2 - DNA, 1340 bp, Arabidopsis thaliana
atgtcgagtttttctgccgagtgttgggacttctctggtcgtaaaccgagcttttcacaaacatgtactcgattgagtcgttacctgaaggagaagggtagttttggagatctgagcttagggatgacatgcaagcccgacgttaatggtaatcaacctttctcttttagatctgttcatttcgtttctgcgaattttgagtttcctgttgcgaattacgaattatgaatcgattacatgttgtctggaaattgaatcgttgtagagaatcgatgattaatggattttgaatcaatttctatatatgtatgtatgtgtatgaatttgtggagatggttctgtgtttggtttaagcataatcctctaaagccataggcctggtttagtcattaagtagattttgttttgcagttgtttgagagattggtgaatgataataatctttacttttggatcaattttcgcaggaggttcacgtcagcctacaatgatgaatctgttcccttgtgaagcttcaggaatggattcttctgctggtcaagaagacattaaaccgaagactatgtttccgagacaatcaagcttttcttcttcctcttcctctgggaccaaagaagatgtacagatgatcaaagagactactaagtatgtgtttatatgtatatagtgaaaagaatcgtctcatatagtaagctatgtttcatcagtttaactaatttttgtgtgattttgaacagatctgtgaagccagagtctcaatctgctccgttgactatattctacggtggtcgagttatggtgtttgatgatttttctgctgagaaagctaaagaagtcattgatttggctaacaaaggaagtgccaaaagcttcacatgtttcacagctgaagtaaacaataaccatagtgcttattctcaaaaagagattgcttctagcccaaatcctgtttgtagtcctgcaaaaaccgcagcacaagagccaattcagcctaacccggcctctttagcctgcggtaattgatctcagcctcttttggttcaaccatctctgagcttggcttagtcctatttctctaactttctctctttttgatttgtagaactcccgattgcaagaagagcttcacttcatcggttccttgagaagaggaaggataggtaatgcattttaatgaaaacataatatcttgttattccataagttcttgttaagcgaatttctcaatatcctactcgtaaacctttaacaggatcacatcaaaggcaccataccaaatagacggttcagctgaagcgtcttccaagcctactaacccagcttggctcagttcacggtaa

SEQ ID NO: 3 - protein, 249 aa, Arabidopsis thaliana
MSSFSAECWDFSGRKPSFSQTCTRLSRYLKEKGSFGDLSLGMTCKPDVNGGSRQPTMMNLFPCEASGMDSSAGQEDIKPKTMFPRQSSFSSSSSSGTKEDVQMIKETTKSVKPESQSAPLTIFYGGRVMVFDDFSAEKAKEVIDLANKGSAKSFTCFTAEVNNNHSAYSQKEIASSPNPVCSPAKTAAQEPIQPNPASLACELPIARRASLHRFLEKRKDRITSKAPYQIDGSAEASSKPTNPAWLSSR

SEQ ID NO: 4 - protein, 28 aa, Artificial Sequence
  Consensus ZIM domain
  misc_feature (1)..(1): Gln, Pro, Arg or Ile
  misc_feature (2)..(2): Leu, Met or Ile
  misc_feature (5)..(5): Phe or Ile
  misc_feature (6)..(6): Tyr or Phe
  misc_feature (7)..(7): Gly, Ala or Asn
  misc_feature (9)..(9): Ser, Lys, Arg, Thr, His or Gln
  misc_feature (10)..(10): Val, Met, Phe or Ile
  misc_feature (11)..(11): Cys, Ile, Leu, Met, Thr or Ser
  misc_feature (12)..(12): Val or Phe
  misc_feature (13)..(13): Phe, Tyr or Ser
  misc_feature (14)..(14): Asp, Asn, Ser or Gln
  misc_feature (15)..(15): Asp, Glu, Gly or Val
  misc_feature (16)..(16): Phe, Ile, Leu, Val or Ser
  misc_feature (17)..(17): Pro, Ser, Thr or Ala
  misc_feature (18)..(18): Ala, Pro, Arg, His, Val, Glu or Ser
  misc_feature (19)..(19): Glu, Asp, Leu or Asn
  misc_feature (20)..(20): Lys, Glu or Gln
  misc_feature (21)..(21): Ala or Val
  misc_feature (22)..(22): Lys, Gln, Gly or Asn
  misc_feature (23)..(23): Glu, Ala or Ser
  misc_feature (24)..(24): Ile or Val
  misc_feature (25)..(25): Met, Leu or Ile
  misc_feature (26)..(26): Leu, Ser, Asn, Asp, Glu, Arg, His or Lys
  misc_feature (27)..(27): Leu, Ile, Val or Cys
XXTIXXXGXXXXXXXXXXXXXXXXXXXA

SEQ ID NO: 5 - protein, 28 aa, Arabidopsis thaliana
PLTIFYGGRVMVFDDFSAEKAKEVIDLA

SEQ ID NO: 6 - protein, 27 aa, Artificial Sequence
  Consensus sequence for Jas domain in Arabidopsis
  MISC_FEATURE (1)..(1): Asp, Glu, Val, Ser or Ala
  MISC_FEATURE (2)..(2): Leu, Val, Glu or Lys
  MISC_FEATURE (3)..(3): Pro, Arg or Ala
  MISC_FEATURE (4)..(4): Ile, Gln, Ser or Leu
  MISC_FEATURE (5)..(5): Ala, Met or Thr
  MISC_FEATURE (6)..(6): Arg or Lys
  MISC_FEATURE (7)..(7): Arg or Lys
  MISC_FEATURE (8)..(8): Ala, Ser, His, Arg or Lys
  misc_feature (9)..(9): Xaa can be any naturally occurring amino acid
  MISC_FEATURE (11)..(11): Gln, His or Ala
  MISC_FEATURE (12)..(12): Arg or Ser
  MISC_FEATURE (14)..(14): Leu or Phe
  MISC_FEATURE (15)..(15): Glu, Ala or Gln
  MISC_FEATURE (18)..(18): Lys, Arg or Ser
  MISC_FEATURE (19)..(19): Asp, Glu, Leu, Ile or His
  MISC_FEATURE (21)..(21): Leu, Ile, Val, Ala or Phe
  MISC_FEATURE (22)..(22): Val, Thr, Ile, Met or Gln
  MISC_FEATURE (23)..(23): ser, Ala, Asn or His
  MISC_FEATURE (24)..(24): Thr, Lys, Arg, Val or Ala
  MISC_FEATURE (25)..(25): Ser, Ala, Asn, Lys or Met
XXXXXXXXXLXXFXXKRXXRXXXXXPY

SEQ ID NO: 7 - protein, 27 aa, Arabidopsis thaliana
ELPIARRASLHRFLEKRKDRITSKAPY

SEQ ID NO: 8 - DNA, 477 bp, Artificial Sequence
  JAZ2DeltaJas cDNA
atgtcgagtttttctgccgagtgttgggacttctctggtcgtaaaccgagcttttcacaaacatgtactcgattgagtcgttacctgaaggagaagggtagttttggagatctgagcttagggatgacatgcaagcccgacgttaatggaggttcacgtcagcctacaatgatgaatctgttcccttgtgaagcttcaggaatggattcttctgctggtcaagaagacattaaaccgaagactatgtttccgagacaatcaagcttttcttcttcctcttcctctgggaccaaagaagatgtacagatgatcaaagagactactaaatctgtgaagccagagtctcaatctgctccgttgactatattctacggtggtcgagttatggtgtttgatgatttttctgctgagaaagctaaagaagtcattgatttggctaacaaaggaagtgccaaaagcttcacatgtttcacatga

SEQ ID NO: 9 - DNA, 888 bp, Artificial Sequence
  Jaz2Deltajas gDNA
atgtcgagtttttctgccgagtgttgggacttctctggtcgtaaaccgagcttttcacaaacatgtactcgattgagtcgttacctgaaggagaagggtagttttggagatctgagcttagggatgacatgcaagcccgacgttaatggtaatcaacctttctcttttagatctgttcatttcgtttctgcgaattttgagtttcctgttgcgaattacgaattatgaatcgattacatgttgtctggaaattgaatcgttgtagagaatcgatgattaatggattttgaatcaatttctatatatgtatgtatgtgtatgaatttgtggagatggttctgtgtttggtttaagcataatcctctaaagccataggcctggtttagtcattaagtagattttgttttgcagttgtttgagagattggtgaatgataataatctttacttttggatcaattttcgcaggaggttcacgtcagcctacaatgatgaatctgttcccttgtgaagcttcaggaatggattcttctgctggtcaagaagacattaaaccgaagactatgtttccgagacaatcaagcttttcttcttcctcttcctctgggaccaaagaagatgtacagatgatcaaagagactactaagtatgtgtttatatgtatatagtgaaaagaatcgtctcatatagtaagctatgtttcatcagtttaactaatttttgtgtgattttgaacagatctgtgaagccagagtctcaatctgctccgttgactatattctacggtggtcgagttatggtgtttgatgatttttctgctgagaaagctaaagaagtcattgatttggctaacaaaggaagtgccaaaagcttcacatgtttcacatga

SEQ ID NO: 10 - protein, 158 aa, Artificial Sequence
  JAZ2DeltaJas aminoacids
MSSFSAECWDFSGRKPSFSQTCTRLSRYLKEKGSFGDLSLGMTCKPDVNGGSRQPTMMNLFPCEASGMDSSAGQEDIKPKTMFPRQSSFSSSSSSGTKEDVQMIKETTKSVKPESQSAPLTIFYGGRVMVFDDFSAEKAKEVIDLANKGSAKSFTCFT

SEQ ID NO: 11 - DNA, 503 bp, Arabidopsis thaliana
ctaaatggtataactgaaagactatcataatcaaatttcatctgaacattgatttgtttgaactaattaattttaggattttacgttttcataattttttattttatttgaacatcttgtaagggtaaaatggtaaaaacaggagtagtaagagggtgaaatggttatttcaagtgtgaagcgtggggacgtgggtgacggtggtgatctgaaacagtggtctgccgttaaaacattaaattaaaacacgtgttttaaatgtctctatgttaattaatactataagagtatattttactaatttccgtatctgcaaccagcgaaaaaaaagtaataaagaggtcctccatttcttcctcgtgacaaaacgcacttggcagagaaagataaacaagaaccctaagtttttttataagattcgagaaaattcaacaactcaggaaggaagatccttttgctccaatttctcaatcgaaacgatttcaatttcggtttcaacgatg

SEQ ID NO: 12 - DNA, 31 bp, Artificial Sequence
  Jaz2.3 trasposon insertion mutant
atgtcgagtttttctgccgagtgttgggact

SEQ ID NO: 13 - DNA, 2021 bp, Arabidopsis thaliana
gatctggctcgacgtcgacgagaagcaggatgagccatcaaaggatccactaaagcaccaaactcaaaaaaaagagagccaaatattgcaactactcaagtcagtctaaatatagttaaatgctgcgctatatatcatgcatatacatatgtcatttgattttctctttgccattatttactctcattcgattttaatacaaaattatttaagttgttgttgtaaacagacgttgctgttgctgttgagtttgatttagaatagttaagtttggttttggttggagaaaagttttcgttgttaagccaactcgtgatcatttaatctgttctttttttggtagggggtgatcattgaatcttttgttttgagttttgtatgtctatatagagtctaataccattaaaacaaaaaaaatgtcgtggtaaccagaattaaatgatgtaaaaacagaaaacaagaattaaatgtcggatgtaaaaacagaattaaatttcggatgttttatgtggatcatagcgtgagagatgcgtttatggcatcttttgatataatcttcgctttgtaggtccacttttgatctgatctgatctattatctatatggaatgttgtgtatataataaactgcagtgtagtattttcttaataatgtgatattaatatctaaattctaggctggagaaaatgacgtgtgctgcatatcccccagactcgaaataacgatgttatcgtgggagatgatcgtcaattttttttttgtttagagatcgtcaagttgttgtgatcattgactcctttccattttttctatagatttgaattttaaatattcattttcataaaaaaatgaaaactttattattatatttgtgagataattagatatgttttaaaacattagtaaatttagtatagtattagtttttactaaattaagaagaaagtgacgcacgaaatggcgtaatttcctcccccggaccacatgcatcttataaatacacaaaaacgatgtcaacgagtgattttagtaatatgtacattaaagtaatattttctttttggtaccaataattaccttaattaatacaagtatagtttaagcaacaagcaacattctttaataattcgcgaaaaatattaataaaagtacaatgacagaaaaaataattaaaaattggattaaaaaaaataatattagagtgatgacataatcacatgaggatttagatttagcttaataatgtttagttgtggaactaaatttccgcaacaattattttgttgtatttaaatcattttatgaaagtctaatctttacaattactttatagtaccatccgattaatcaaagattaaaaatatagtactaaaatacaaattaatatattttgccaaaaaaatgcaacgtgcaaatgtttatcttggtcaagagaacgtgcacgaaagtagtgtaactaattttactattttagattgagcggtgagacaaaaagggggttagactcacgcgctacacttagcgcgtcagccgacaaaaaaaagtgttgtcattgaataaaaaaataaattaaaactgccgcttcgcatctcgtgaatcattcacgttaaaatagcgcacgtgcctcgccacgcgtctcaccacaaaaagaaacgccacacactgcacgacgatgcacgtgcctttttaaccgactatatccggtgacgtaaccgggacaacctgtagctggtttttgcgccaataggatatgagaaaaaagaatcttttttttcttgttatagttataaattaatggtacgattcacatgtcgcatgcgatttgttaataaaactagaaattgcggtgaattaacttcattccacgttttttcattttctccctcaaaagtctctgttttttttcctttttccggcgaagctctatttagcttgattccggcgtttaacacgcgttttaatcgaaacagacatttgagatcgaattaattttgtagcgggctgtgtctttattatagatg

SEQ ID NO: 14 - DNA, 894 bp, Arabidopsis thaliana
atggagagagattttctcgggttgggttcgaaaaattctccgatcactgtcaaggaggaaaccagcgaaagctctagagattcagctcccaacagaggaatgaactggtctttctcaaacaaagtatcagcttcttcttctcagtttctatccttcaggccaactcaagaagatagacatagaaagtctggaaattatcatcttcctcactctggttccttcatgccatcatcagtagctgatgtttatgattcaacccgcaaagctccttacagttctgtacagggagtgaggatgttccctaattccaatcaacacgaagaaactaacgcagtttccatgtcgatgccgggtttccagtctcatcattatgcaccaggaggaagaagcttcatgaacaataacaataactcacaacctttggtaggagttcctatcatggcacctccaatttcaatccttcctcctccaggttccattgtagggactactgatattagatcttcttccaagccaataggttcacctgcgcagttgacgatcttttatgccggttcagtttgtgtttacgatgacatatctcctgaaaaggcaaaggcgataatgttgctagctgggaacggttcctctatgcctcaagtcttttcgccgcctcaaactcatcaacaagtggtccatcatactcgtgcctctgtcgattcttcagctatgcctcctagcttcatgcctacaatatcttatcttagccctgaagctggaagtagcacaaacggactcggagccacaaaagcgacaagaggcttgacgtcaacatatcacaacaaccaagctaatggatccaatattaactgcccagtaccagtttcttgttctaccaatgtaatggctccaaca

SEQ ID NO: 15 - protein, 312 aa, Artificial Sequence
  JAZ3DeltaJas
MERDFLGLGSKNSPITVKEETSESSRDSAPNRGMNWSFSNKVSASSSQFLSFRPTQEDRHRKSGNYHLPHSGSFMPSSVADVYDSTRKAPYSSVQGVRMFPNSNQHEETNAVSMSMPGFQSHHYAPGGRSFMNNNNNSQPLVGVPIMAPPISILPPPGSIVGTTDIRSSSKPIGSPAQLTIFYAGSVCVYDDISPEKAKAIMLLAGNGSSMPQVFSPPQTHQQVVHHTRASVDSSAMPPSFMPTISYLSPEAGSSTNGLGATKATRGLTSTYHNNQANGSNINCPVPVSCSTNVMAPTGKKQSQRPDTTFAI

SEQ ID NO: 16 - protein, 28 aa, Arabidopsis thaliana
PLTIFYAGQVIVFNDFSAEKAKEVINLA

SEQ ID NO: 17 - protein, 28 aa, Arabidopsis thaliana
QLTIFYAGSVCVYDDISPEKAKAIMLLA

SEQ ID NO: 18 - protein, 28 aa, Arabidopsis thaliana
QLTIFYAGSVLVYQDIAPEKAQAIMLLA

SEQ ID NO: 19 - protein, 28 aa, Arabidopsis thaliana
QLTIFFGGKVLVYNEFPVDKAKEIMEVA

SEQ ID NO: 20 - protein, 28 aa, Arabidopsis thaliana
QLTIFFGGKVMVFNEFPEDKAKEIMEVA

SEQ ID NO: 21 - protein, 28 aa, Arabidopsis thaliana
ILTIFYNGHMCVSSDLTHLEANAILSLA

SEQ ID NO: 22 - protein, 28 aa, Arabidopsis thaliana
RITIFYNGKMCFSSDVTHLQARSITSIA

SEQ ID NO: 23 - protein, 28 aa, Arabidopsis thaliana
QLTIFYGGTISVFNDISPDKAQAIMLCA

SEQ ID NO: 24 - protein, 27 aa, Arabidopsis thaliana
PMTIFYNGSVSVFQVSRNKAGEIMKVA

SEQ ID NO: 25 - protein, 28 aa, Arabidopsis thaliana
QLTIIFGGSFSVFDGIPAEKVQEILHIA

SEQ ID NO: 26 - protein, 28 aa, Arabidopsis thaliana
QLTIFFGGSVTVFDGLPSEKVQEILRIA

SEQ ID NO: 27 - protein, 4 aa, Artificial Sequence
  Consensus TiFY
  VARIANT (3)..(3): Thr or conserved amino adic
  VARIANT (4)..(4): Tyr or conserved amino adic
TIXX

SEQ ID NO: 28 - protein, 4 aa, Artificial Sequence
  TIFY domain
TIFY

SEQ ID NO: 29 - DNA, 24 bp, Artificial Sequence
  forward primer pJAZ1 fw
caccgtacgttccaacttctaacc

SEQ ID NO: 30 - DNA, 28 bp, Artificial Sequence
  reverse primer pJAZ1 rev
catctttaacaattaaaactttcaaacg

SEQ ID NO: 31 - DNA, 25 bp, Artificial Sequence
  forward primer pJAZ2 fw
caccgactaagaatttgttatgaag

SEQ ID NO: 32 - DNA, 26 bp, Artificial Sequence
  reverse primer pJAZ2 rev
catcgttgaaaccgaaattgaaatcg

SEQ ID NO: 33 - DNA, 30 bp, Artificial Sequence
  forward primer pJAZ3 fw
caccgatctggctcgacgtcgacgagaagc

SEQ ID NO: 34 - DNA, 25 bp, Artificial Sequence
  reverse primer pJAZ3 rev
catctataataaagacacagcccgc

SEQ ID NO: 35 - DNA, 16 bp, Artificial Sequence
  forward primer AtANAC19_fw
gcatctcgtcgctcag

SEQ ID NO: 36 - DNA, 17 bp, Artificial Sequence
  reverse primer AtANAC19_rev
ctcgacttcctcctccg

SEQ ID NO: 37 - DNA, 16 bp, Artificial Sequence
  forward primer AtANAC55_fw
gcgctgcctcatagtc

SEQ ID NO: 38 - DNA, 17 bp, Artificial Sequence
  reverse primer AtANAC55_rev
cgaggaatcccctcagt

SEQ ID NO: 39 - DNA, 17 bp, Artificial Sequence
  forward primer AtANAC72_fw
tgggtgttgtgtcgaat

SEQ ID NO: 40 - DNA, 18 bp, Artificial Sequence
  reverse primer AtANAC72_rev
atcgtaaccaccgtaact

SEQ ID NO: 41 - DNA, 21 bp, Artificial Sequence
  forward primer AtACTIN8_fw
ccagtggtcgtacaaccggta

SEQ ID NO: 42 - DNA, 24 bp, Artificial Sequence
  reverse primer AtACTIN8_rev
tagttcttttcgatggaggagctg

SEQ ID NO: 43 - DNA, 20 bp, Artificial Sequence
  AtJAZ2 Full Length primer Forward (AtJAZ2 FL_F)
cgagtgttgggacttctctg

SEQ ID NO: 44 - DNA, 21 bp, Artificial Sequence
  AtJAZ2 Full Length Primer Reverse (AtJAZ2 FL_R)
gctcttcttgcaatcgggagt

SEQ ID NO: 45 - DNA, 20 bp, Artificial Sequence
  AtJAZ2-3 Forward primer (AtJAZ2-3_F)
tggattcttctgctggtcaa

SEQ ID NO: 46 - DNA, 20 bp, Artificial Sequence
  AtJAZ2DeltaJas Forward primer (JAZ2DeltaJas_F)
tgcttctagcccaaatcctg

SEQ ID NO: 47 - DNA, 20 bp, Artificial Sequence
  AtJAZ2DeltaJas Reverse primer (JAZ2DeltaJas_R)
tctttggtcccagaggaaga

SEQ ID NO: 48 - DNA, 20 bp, Artificial Sequence
  AtPR1 Forward primer
gtgggttagcgagaaggcta

SEQ ID NO: 49 - DNA, 20 bp, Artificial Sequence
  AtPR1 Reverse primer
actttggcacatccgagtct

SEQ ID NO: 50 - DNA, 22 bp, Artificial Sequence
  AtPR2_Forward primer
gtctgaatcaaggagcttagcc

SEQ ID NO: 51 - DNA, 21 bp, Artificial Sequence
  AtPR2 reverse primer
ccgtagcatactccgatttgt

SEQ ID NO: 52 - DNA, 21 bp, Artificial Sequence
  AtANAC19 G-box Forward primer
agcagctactacgagttgtgt

SEQ ID NO: 53 - DNA, 20 bp, Artificial Sequence
  At ANAC19 G-box Reverse primer
cgtgtccacgtgtctatcgt

SEQ ID NO: 54 - DNA, 20 bp, Artificial Sequence
  At ANAC55 G-box Forward primer
tgtgtcggcttgtggtagtt

SEQ ID NO: 55 - DNA, 21 bp, Artificial Sequence
  At ANAC55 G-box Rreverse primer
gggatgagttcactggatggt

SEQ ID NO: 56 - DNA, 20 bp, Artificial Sequence
  At ANAC72 G-box Forward primer
tgcaatcactcagcggactt

SEQ ID NO: 57 - DNA, 20 bp, Artificial Sequence
  At ANAC72 G-box Rreverse primer
ggccgaccttatcgatgtgt

SEQ ID NO: 58 - DNA, 25 bp, Artificial Sequence
  AtACTIN8 ChIP Forward primer
gactcagatcatgtttgagaccttt

SEQ ID NO: 59 - DNA, 20 bp, Artificial Sequence
  AtACTIN8 ChIP Reverse primer
accggttgtacgaccactgg

SEQ ID NO: 60 - protein, 253 aa, Arabidopsis thaliana
MSSSMECSEFVGSRRFTGKKPSFSQTCSRLSQYLKENGSFGDLSLGMACKPDVNGTLGNSRQPTTTMSLFPCEASNMDSMVQDVKPTNLFPRQPSFSSSSSSLPKEDVLKMTQTTRSVKPESQTAPLTIFYAGQVIVFNDFSAEKAKEVINLASKGTANSLAKNQTDIRSNIATIANQVPHPRKTTTQEPIQSSPTPLTELPIARRASLHRFLEKRKDRVTSKAPYQLCDPAKASSNPQTTGNMSWLGLAAEI

SEQ ID NO: 61 - protein, 195 aa, Artificial Sequence
  JAZ1DeltaJas
MSSSMECSEFVGSRRFTGKKPSFSQTCSRLSQYLKENGSFGDLSLGMACKPDVNGTLGNSRQPTTTMSLFPCEASNMDSMVQDVKPTNLFPRQPSFSSSSSSLPKEDVLKMTQTTRSVKPESQTAPLTIFYAGQVIVFNDFSAEKAKEVINLASKGTANSLAKPISEATSLLSQTKFLIQEKPQHKSQSNPPQHH

SEQ ID NO: 62 - DNA, 25 bp, Artificial Sequence
  SlActin forward primer
caagttattaccattggtgctgaga

SEQ ID NO: 63 - DNA, 22 bp, Artificial Sequence
  SlActin Reverse primer
tgcagcttccataccaatcatg

SEQ ID NO: 64 - DNA, 21 bp, Artificial Sequence
  SlJaz1 Forward primer
ggaaacaatcctgctaaacca

SEQ ID NO: 65 - DNA, 18 bp, Artificial Sequence
  SlJaz1 Reverse primer
tccgaaactcggaaccac

SEQ ID NO: 66 - DNA, 23 bp, Artificial Sequence
  SlJaz2 Forward primer
aagacagaatcttggaaacctga

SEQ ID NO: 67 - DNA, 24 bp, Artificial Sequence
  SlJaz2 Reverse primer
aacaatgacttgtccaccataaaa

SEQ ID NO: 68 - DNA, 23 bp, Artificial Sequence
  SlJaz3 Forward primer
aacacctccagattaagccagac

SEQ ID NO: 69 - DNA, 20 bp, Artificial Sequence
  SlJaz3 Reverse primer
aattgtgcttgtgctgttgc

SEQ ID NO: 70 - DNA, 26 bp, Artificial Sequence
  SlJaz4 Forward primer
tggaaaagcaaatatcaatgatctaa

SEQ ID NO: 71 - DNA, 21 bp, Artificial Sequence
  SlJAZ4 Reverse primer
acaaatcctttgttgctgagg

SEQ ID NO: 72 - DNA, 24 bp, Artificial Sequence
  gRNA for SlJAZ2 (A)
attgaaatcagcaacagaaggctg

SEQ ID NO: 73 - DNA, 24 bp, Artificial Sequence
  gRNA for SlJAZ2 (B)
aaaccagccttctgttgctgattt

SEQ ID NO: 74 - DNA, 24 bp, Artificial Sequence
  2 gRNA for SlJAZ2 (A)
attgctgatttaccaatcgcgaga

SEQ ID NO: 75 - DNA, 24 bp, Artificial Sequence
  2 gRNA for SlJAZ2 (B)
aaactctcgcgattggtaaatcag

SEQ ID NO: 76 - DNA, 24 bp, Artificial Sequence
  SlJaz2 Forward primer
atcatgaagttagccaacaaacag

SEQ ID NO: 77 - DNA, 26 bp, Artificial Sequence
  SlJaz2 Reverse primer
gaaatattgctcagttttaacaaatt

SEQ ID NO: 78 - protein, 216 aa, Solanum lycopersicum
MASSEIVDSGRFAGQKSHFSHTCNLLSQYLKEKKGSLGDLSLDMHRNFDSAGSTTMDLLPMIEKSGELVQKSMNLFPQGGMKAESEPEKAQMTIFYGGQVIVFNDFPADKAKEIMLMASTSKGNNPAKPLESAADLVVPSFGKTSIQENQMPNQPIVSDLPIARRASLTRFLEKRKDRLTAKVPYHREEAAAPKKEEHKAPWLGLGGQFAVKTEQY

SEQ ID NO: 79 - protein, 252 aa, Solanum lycopersicum
MGSSENMDSGKVTGQKSQFSQTCNLLSQFLKKKGSVGDLNNLGIYKTTFESTGSQQTATTTTTTMNLLPMIEKSSDSSSSSSSVETNPQKPMNLFPQEFDFSKEQSTKKTESWKPDQPEKAQMTIFYGGQVIVFDDFPADKANEIMKLANKQNPTNNFTYPMIKNQKTADQSGVSFGNKLIQELPKLSMPQPSVADLPIARRNSLTRFLEKRKDRVTSIAPYQISNNKKSKNEDNKAWLGLGAQFVKTEQYF

SEQ ID NO: 80 - protein, 309 aa, Solanum lycopersicum
MSNLCDARRRNGNGKAPERSSFVQTCNLLSQFIKGKATIRDLNLGIAGQPEAAGKTETATMDLLTVMEKPSIDLTKEEHKSVDLVTTESSREKEAAVNEPSTSKEAPKEPKAAQLTMFYDGKVIVFDDFPADKARAVMLLASKGCPQSSFGTFQAINIDKINTCSPAPASLTSNRTDSVAPQQQHLQIKPDSCSAAPQQHKHNSPPLHVCSSTKTDQLKLGSVSSAPLVEQEQHKQIQSQAAEISSSSELPIARRSSLHRFLEKRKDRATVRAPYQVVRNNPLLPSSSNTNGESSSKDSEDQLDLNFKL

SEQ ID NO: 81 - protein, 200 aa, Solanum lycopersicum
MSNRQLCSLDSEKSHLMNTCNLLTQFFNGKANINDLNLTISNNGEAKASATKDLLTNMEELSTKTTEQDQKLIDHVPKSAINKASGSKEIPHKEQKLAQLSIFYGGKVVVFDDFPAEKARAVMLLASKGISNNSCAIFQTPTTTQTNGSNNFDLPIARRSSLYRFLEKRKDRDTARAPYQMHNPLQSSSRTRGDHFDLNF

SEQ ID NO: 82 - DNA, 2063 bp, Solanum lycopersicum
  misc_feature (1855)..(1909): Jas domain
atggggtcatcggaaaatatggattccggcaaggtgaccggacagaaatctcagttttctcagacttgtaaccttttgagtcaattcttgaagaaaaaaggttctgttggagatcttaataatcttggtatctacaaaactacttttgaatcaactggtatgttcttttttaaatttttttttgaactttatttatgtttttatgaatcattatttgctattataatgtttttggttgtaaagattgaagctttattggttttgggagtatgtattcatgtttcttgttgcaaagatagtactctggttggttttgtgaaacttttcgagtttcgagatttgaataagcgtatgtttgaccgtaaagttttcatatatcttttaattattttgagttatagtaaactttataaatttcattttaaaaactctatagattccgggcataagttctcggtcaaacttaaactatttggcttttagaaaacgaacggtgtcacgtaaattgagacgagaaaaactgtttttggttgttgaaaggatttgttcatgtttctggttgcaagattgtagcttttttggttttggggagtatatgttcatgtttgttgttgcaaagattgtagcttttgggatttttgaaaggattttttcatgtttatggttgcaaagattgtagcttttgggttttctttgaagtatttattgttcgaaaacaacctctctgtctgttaagatctgtgtataatctattcttcctatagcacctagcgagatttcactgggtatattgttgttgtatttttggttgcaatgacttagagtaggtttggtcgtagattttgaagttgaaacttttgagtttttgaagttttgtttggatgcgtgttttacttgaaactaattgaagtgttgtgagagaaagaagaaagtccgggattagtttttggaaacttgaaaatattcgaagatctgataatatttcataatcaagtagatatttgacgattaaatttcaaaatctatgtttttgttgcaaagatgtgtcacaagtctattaacaagttcgacataactcaataattttggttaaaactttttatttgtgtttaatttttttacttaatatgtaaatagtactaattaatagaacattaaactatcttttctgaacttcataactcataaaatcgaaatcctaactccgcctctccctttttggtttttagaagtagtttttcatgtttctaaatccaaagagctactagtttgtctacaagttcgacacagctcaatagctttgatctaaacttaaaaactttaaaacacaaatctcgaaaatcctcataaactcgaaattctaattctgctatgttctttatgtttctggttacaaagattgtaattttttgtgttttggtttatgaaactgattgaactttccatgtttcttgaaaacaggatctcaacaaacagctactaccaccacaactactatgaatttgttaccaatgattgagaaatcaagtgattcatcgtcgtcatcgtcgtccgttgaaacaaatcctcaaaaacccatgaatcttttccctcaagaatttgatttctccaaagaacaatccacaaaaaagacagaatcttggaaacctgatcaaccagagaaggcacaaatgaccattttttatggtggacaagtcattgtttttgatgattttccagctgataaagcaaatgaaatcatgaagttagccaacaaacagaaccccacaaacaatttcacttatcctatgatcaagaatcaaaaaacagctgatcaatctggtgtgagttttggtaacaaattgattcaagaacttcctaaactgtcaatgccacagccttctgttgctgatttaccaatcgcgagacggaattcacttacaagattcttggagaaaaggaaagatagagtaacatcaattgcaccataccaaatctccaacaacaagaaatccaagaatgaggataacaaagcatggttgggattaggtgctcaatttgttaaaactgagcaatatttctag

SEQ ID NO: 83 - DNA, 48 bp, Artificial Sequence
  Jas Domain in mutant 2.3.2
ttcctaaactgtcaatgccacagttgctgatttaccaatcgcgagacg

SEQ ID NO: 84 - DNA, 51 bp, Artificial Sequence
  Jas domain mutant 15.1.2
ttcctaaactgtcaatgccacagctgttgctgatttaccaatcgcgagacg